(19) United States
(12) Patent Application Publication
(10) Pub. No.: US 2025/0064335 A1
(43) Pub. Date: Feb. 27, 2025

(54) ELECTRICAL IMPEDANCE TOMOGRAPHY BASED DIAGNOSTIC SYSTEMS AND METHODS

(71) Applicant: Gense Technologies Limited, Hong Kong (CN)

(72) Inventors: Russell Wade Chan, Kowloon (HK); Adrien Touboul, Wan Chai (HK); Chung San Wong, Tseung Kwan O (HK); Fedi Zouari, Tseung Kwan O (HK); Dipyaman Modak, Mong Kok (HK); Pak Heng Justin Chan, Pok Fu Lam (HK); Pak To Cheung, North Point (HK); Ho Wa Li, Sheung Shui, New Territories (HK)

(21) Appl. No.: 18/726,191

(22) PCT Filed: Jan. 28, 2023
(86) PCT No.: PCT/CN2023/073608

§ 371 Date: Jul. 2, 2024

(30) Foreign Application Priority Data
Jan. 24, 2022 (HK) 32022046981.3

(51) Int. Cl. A61B 5/0536; A61B 5/00; A61B 5/20; G16H 30/40

(52) U.S. Cl. CPC A61B 5/0536; A61B 5/4842; A61B 5/7267; G16H 30/40; A61B 5/201

(57) ABSTRACT

A computer-implemented method that includes processing a EIT data set of a subject to determine one or more conductivity characteristics associated with a tissue or organ of the subject, and, determining, based on at least the one or more determined conductivity characteristics, a health state or condition of the tissue or organ of the subject.

### TECHNICAL FIELD

The invention relates to electrical impedance tomography (EIT) based diagnostic systems and methods.

### BACKGROUND

Electrical impedance tomography is a medical imaging technique that can be used for determining electrical conductivity, permittivity, and/or impedance of a body part of a subject (animal, human, etc.).

### SUMMARY OF THE INVENTION

In a first aspect, there is provided a computer-implemented method, comprising: processing a EIT data set of a subject to determine one or more conductivity characteristics associated with a tissue or organ of the subject; and determining, based on at least the one or more determined conductivity characteristics, a health state or condition of the tissue or organ of the subject.

Optionally, the determining comprises: determining, based on at least the one or more determined conductivity characteristics, whether the subject has a disease associated with the tissue or organ, and optionally: further classifying a stage or a severity of the disease associated with the tissue or organ.

Optionally, the determining comprises: processing, at least, the one or more determined conductivity characteristics of the subject and one or more anthropometric characteristics of the subject, using a machine learning based processing model, to determine a quantitative or qualitative parameter associated with the health state or condition of the tissue or organ of the subject.

Optionally, the determining comprises: processing, using a machine learning based processing model, (i) the one or more determined conductivity characteristics of the subject, (ii) one or more anthropometric characteristics of the subject, and (iii) one or more determined conductivity characteristics of one or more reference subjects and/or of a group containing the subject and the one or more reference subjects, to determine a quantitative or qualitative parameter associated with the health state or condition of the tissue or organ of the subject.

Optionally, the machine learning based processing model comprises a regression model. Optionally, the machine learning based processing model comprises a classification model. The machine learning based processing model may be recurrent models or non-recurrent models. The machine learning based processing model may include, e.g., recursive neural network, recurrent neural network, long-short term memory model, Markov process, reinforcement learning, gated recurrent unit model, deep neural network, convolutional neural network, support vector machines, principle component analysis, logistic regression, decision trees/forest, ensemble method (combining model), regression (Bayesian/polynomial/regression), stochastic gradient descent, linear discriminant analysis, nearest neighbor classification or regression, naive Bayes, etc. The machine learning based processing model can be trained to perform a particular processing or classification task associated with the diagnostic application.

Optionally, the one or more anthropometric characteristics comprise, or are related to, one or more of: age of the subject, weight of the subject, height of the subject, waist circumference of the subject, waist-over-height ratio of the subject, body mass index (BMI) of the subject, gender of the subject, and race of the subject. Other anthropometric characteristic(s) are possible.

Optionally, the quantitative or qualitative parameter associated with the health state or condition of the tissue or organ of the subject comprises: a value associated with an estimated performance of the tissue or organ of the subject. For example, in respect of kidney, the value may be an estimated glomerular filtration rate (eGFR) or a related value (e.g., arithmetically related). For example, in respect of liver, the value may be a controlled attenuation parameter (CAP) score or a related value (e.g., arithmetically related).

Optionally, the determining further comprises: comparing the quantitative or qualitative parameter associated with the health state or condition of the tissue or organ of the subject with reference parameter data to determine whether the subject has a disease associated with the tissue or organ.

Optionally, the determining further comprises: classifying, based on the comparing, a stage or a severity of the disease associated with the tissue or organ.

Optionally, the EIT data set contains EIT data obtain from a region of the subject containing the tissue or organ. Optionally, the EIT data set is obtained by (a) providing excitation signals at a frequency to the subject via electrodes attached to the region of the subject, (b) measuring responsive signals received via the electrodes as a result of the providing of the excitation signals, and (c) repeating steps (a) and (b) for a plurality of frequencies. The EIT data set comprises a plurality of EIT data subsets each associated with a respective one of the plurality of frequencies.

Optionally, the processing comprises: (i) processing the EIT data set to obtain a frequency difference EIT data set, the frequency difference EIT data set includes a plurality of frequency difference EIT data subsets; (ii) performing a group source separation operation using the frequency difference EIT data set and one or more reference frequency difference EIT data sets of corresponding one or more reference subjects to determine component of the frequency difference EIT data set related to the tissue or organ and component of each of the one or more reference frequency difference EIT data sets related to the tissue or organ; and (iii) performing a conductivity characteristics extraction operation using the component of the frequency difference EIT data set related to the tissue or organ and optionally the component of each of the one or more reference frequency difference EIT data sets related to the tissue or organ to determine at least the one or more conductivity characteristics of the subject.

Optionally, the processing further comprises: pre-processing the EIT data set before the processing in (i) so that the EIT data set processed in (i) is a pre-processed EIT data set.

Optionally, the pre-processing of the EIT data set comprises: filtering and/or smoothing each of the plurality of EIT data subsets.

Optionally, the pre-processing of the EIT data set comprises: processing the EIT data set using a classifier model to determine respective performance of each of the plurality of electrodes, the performance being associated with quality of responsive signals or data obtained from the respective electrode; and preventing the responsive signals or data obtained via any one or more of the plurality of electrodes determined to have insufficient performance from being included in the processed EIT data set.

Optionally, the processing of the EIT data set in (ii) comprises: determining, for each respective one or more of the plurality of processed EIT data subsets, respective difference between the respective processed EIT data subset and a reference EIT data subset, so as to obtain the plurality of frequency difference EIT data subsets each associated with a respective one of a difference between the respective processed EIT data subset and a reference EIT data subset.

Optionally, the reference EIT data subset comprises at least one of the plurality of processed EIT data subsets.

Optionally, the performing of the group source separation operation comprises: performing a dimensionality reduction operation on the frequency difference EIT data set and one or more reference frequency difference EIT data sets of corresponding one or more reference subjects.

Optionally, the performing of the conductivity characteristics extraction operation comprises: determining, using the component of the frequency difference EIT data set related to the tissue or organ, the one or more conductivity characteristics of the subject. The one or more conductivity characteristics of the subject may include one or more statistical conductivity characteristics of the subject (e.g., mean, median, mode, standard deviation, etc., of any part of the fd-EIT data or EIT data of the subject (that can be represented as conductivity map or image).

Optionally, the performing of the conductivity characteristics extraction operation comprises: determining, using the component of the frequency difference EIT data set related to the tissue or organ and respective component of each of the one or more reference frequency difference EIT data sets related to the tissue or organ, one or more conductivity characteristics of a group containing the subject and the one or more reference subjects. The determining of the health state or condition of the tissue or organ of the subject is further based on the one or more conductivity characteristics of the group. The one or more conductivity characteristics of the subject may include one or more statistical conductivity characteristics of the group (e.g., mean, median, mode, standard deviation, etc., of any part of the fd-EIT data or EIT data of the group (that can be represented as conductivity map or image).

Optionally, the determining of the health state or condition of the tissue or organ of the subject is further based on the one or more conductivity characteristics of the one or more reference subjects. The one or more conductivity characteristics of the one or more reference subjects may include one or more statistical conductivity characteristics of the group (e.g., mean, median, mode, standard deviation, etc., of any part of the fd-EIT data or EIT data of the one or more reference subjects (that can be represented as conductivity map or image).

Optionally, the tissue or organ comprises a lung, a kidney, a liver, or a heart.

In a second aspect, there is provided a system comprising one or more processors and memory storing one or more programs configured to be executed by the one or more processors. The one or more programs include instructions for performing or facilitating performing of the computer-implemented method of the first aspect.

In a third aspect, there is provided a non-transitory computer-readable storage medium storing one or more programs configured to be executed by one or more processors. The one or more programs include instructions for performing or facilitating performing of the computer-implemented method of the first aspect.

In a fourth aspect, there is provided a computer program product comprising instructions which, when the computer program is executed by a computer, cause or facilitate the computer to carry out the computer-implemented method of the first aspect.

In a fifth aspect, there is provided a computer-implemented method, comprising: processing electrical impedance tomography data obtained from a subject, the electrical impedance tomography data including a plurality of electric potential data sets, each electric potential data set being obtained at electrodes attached (directly or indirectly) to the subject in response to excitation signal (e.g., current) of a set frequency sequentially applied to each of the electrodes, the set frequency applied is different for different data sets and is the same of the same data set; and determining, based on the processing, whether the subject has a disease.

Optionally, the plurality of electric potential data sets comprises, at least, a first electric potential data set associated with excitation signal (e.g., current) of a first frequency, a second electric potential data set associated with excitation signal (e.g., current) of a second frequency, and a third electric potential data set associated with excitation signal (e.g., current) of a third frequency. Optionally, the processing comprises: determining a difference between the first and second electric potential data sets to obtain a first electric potential difference data set; determining a difference between the first and third second electric potential data sets to obtain a second electric potential difference data set; applying the first and second electric potential difference data sets to a spectral unmixing model to determine parameters indicative of impact caused by tissue type i on the first and second electric potential difference data sets; and determining a value of a parameter associated with the disease based on the first and second corrected electric potential difference data sets and one or more anthropometric measures of the subject. In this example, the first electric potential data set is used as a reference data set. The reference data set may have the highest signal to noise ratio among all the data sets.

Optionally, the spectral unmixing model includes ΔV(ω)=Σᵢ₌₁^{M} αᵢΔσᵢ(ω)+∈, where ΔV(ω) is the first and second electric potential difference data sets, αᵢ is the parameter indicative of impact caused by tissue type i, Δσᵢ(ω) is predetermined spectrum specific to tissue type i, M is the total number of tissue types, ∈ is an error term. In one example the error term is 0, in which case the spectral unmixing model includes ΔV(ω)=Σᵢ₌₁^{M} αᵢΔσᵢ(ω), where ΔV(ω) is the first and second electric potential difference data sets, αᵢ is the parameter indicative of impact caused by tissue type i, Δσᵢ(ω) is predetermined spectrum specific to tissue type i, M is the total number of tissue types.

Optionally, the plurality of electric potential data sets comprises, at least, a first electric potential data set associated with excitation signal (e.g., current) of a first frequency, a second electric potential data set associated with excitation signal (e.g., current) of a second frequency, and a third electric potential data set associated with excitation signal (e.g., current) of a third frequency, a fourth electric potential data set associated with excitation signal (e.g., current) of a fourth frequency. Optionally, the processing comprises: determining a difference between the first and second electric potential data sets to obtain a first electric potential difference data set; determining a difference between the first and third second electric potential data sets to obtain a second electric potential difference data set; determining a difference between the first and fourth second electric potential data sets to obtain a third electric potential difference data set; applying the first, second, and third electric potential difference data sets to a spectral unmixing model to determine parameters indicative of impact caused by tissue type i on the first, second, and third electric potential difference data sets; and determining a value of a parameter associated with the disease based on the first, second, and third corrected electric potential difference data sets and one or more anthropometric measures of the subject.

Optionally, the spectral unmixing model includes ΔV(ω)=Σᵢ₌₁^{M} αᵢΔσᵢ(ω)+∈, where ΔV(ω) is the first, second, and third electric potential difference data sets, αᵢ is the parameter indicative of impact caused by tissue type i, Δσᵢ(ω) is predetermined spectrum specific to tissue type i, M is the total number of tissue types, ∈ is an error term. In one example the error term is 0, in which case the spectral unmixing model includes ΔV(ω)=Σᵢ₌₁^{M} αᵢΔσᵢ(ω), where ΔV(ω) is the first and second electric potential difference data sets, αᵢ is the parameter indicative of impact caused by tissue type i, Δσᵢ(ω) is predetermined spectrum specific to tissue type i, M is the total number of tissue types.

Optionally, determining whether the subject has a disease includes comparing the determined value with a predetermined reference scale. The predetermined reference scale may include predetermined values of the parameter classified according to presence or absence of the disease, and optionally, severity of the disease.

Optionally, the first electric potential difference data set can be processed to provide a conductivity change map (e.g., average conductivity change map) of the subject. Optionally, the second electric potential difference data set can be processed to provide a conductivity change map (e.g., average conductivity change map) of the subject.

Optionally, the parameter associated with the disease comprises a controlled attenuation parameter.

Optionally, the one or more anthropometric measures of the subject comprises a waist circumference over height (i.e., waist circumference of the subject divided by height of the subject) measure. Optionally, the one or more anthropometric measures of the subject comprises age of the subject. Optionally, the one or more anthropometric measures of the subject comprises chest circumference of the subject.

Optionally, the processing further comprises filtering the electric potential data sets prior to determining the differences. The filtering may remove outlier(s).

Optionally, the computer-implemented method further comprises obtaining the electrical impedance tomography data from the subject.

Optionally, the disease comprises a liver disease, a lung disease, a kidney disease, etc. In one example, the disease comprises a fatty liver disease (e.g., nonalcoholic fatty liver disease).

Optionally, the computer-implemented method further comprises determining, based on the processing, a severity of the disease.

Optionally, the computer-implemented method further comprises presenting the determination result to the user. The presenting may include displaying the result to the user. The result may include a “yes/no” result (as to whether the subject has a disease) and optionally a severity of the disease.

Optionally, the subject is human being. Optionally the subject is a non-human animal.

In a sixth aspect, there is provided a system, comprising: one or more processors arranged to process electrical impedance tomography data obtained from a subject, the electrical impedance tomography data including a plurality of electric potential data sets, each electric potential data set being obtained at electrodes attached (directly or indirectly) to the subject in response to excitation signal (e.g., current) of a set frequency sequentially applied to each of the electrodes, the set frequency applied is different for different data sets and is the same of the same data set; and determine, based on the processing, whether the subject has a disease.

Optionally, the plurality of electric potential data sets comprises, at least, a first electric potential data set associated with excitation signal (e.g., current) of a first frequency, a second electric potential data set associated with excitation signal (e.g., current) of a second frequency, and a third electric potential data set associated with excitation signal (e.g., current) of a third frequency. Optionally, the one or more processors are arranged to: determine a difference between the first and second electric potential data sets to obtain a first electric potential difference data set; determine a difference between the first and third second electric potential data sets to obtain a second electric potential difference data set; apply the first and second electric potential difference data sets to a spectral unmixing model to determine parameters indicative of impact caused by tissue type i on the first and second electric potential difference data sets; and determine a value of a parameter associated with the disease based on the first and second corrected electric potential difference data sets and one or more anthropometric measures of the subject. In this example, the first electric potential data set is used as a reference data set. The reference data set may have the highest signal to noise ratio among all the data sets.

Optionally, the spectral unmixing model includes ΔV(ω)=Σᵢ₌₁^{M} αᵢΔσᵢ(ω)+∈, where ΔV(ω) is the first and second electric potential difference data sets, α₁ is the parameter indicative of impact caused by tissue type i, Δσᵢ(ω) is predetermined spectrum specific to tissue type i, M is the total number of tissue types, e is an error term. In one example the error term is 0, in which case the spectral unmixing model includes ΔV(ω)=Σᵢ₌₁^{M} αᵢΔσᵢ(ω), where ΔV(ω) is the first and second electric potential difference data sets, αᵢ is the parameter indicative of impact caused by tissue type i, Δσᵢ(ω) is predetermined spectrum specific to tissue type i, M is the total number of tissue types. The system may include a memory that stores the spectral unmixing model and is operably connected with the one or more processors.

Optionally, the plurality of electric potential data sets comprises, at least, a first electric potential data set associated with excitation signal (e.g., current) of a first frequency, a second electric potential data set associated with excitation signal (e.g., current) of a second frequency, and a third electric potential data set associated with excitation signal (e.g., current) of a third frequency, a fourth electric potential data set associated with excitation signal (e.g., current) of a fourth frequency. Optionally, the one or more processors are arranged to: determine a difference between the first and second electric potential data sets to obtain a first electric potential difference data set; determine a difference between the first and third second electric potential data sets to obtain a second electric potential difference data set; determine a difference between the first and fourth second electric potential data sets to obtain a third electric potential difference data set; apply the first, second, and third electric potential difference data sets to a spectral unmixing model to determine parameters indicative of impact caused by tissue type i on the first, second, and third electric potential difference data sets; and determine a value of a parameter associated with the disease based on the first, second, and third corrected electric potential difference data sets and one or more anthropometric measures of the subject.

Optionally, the spectral unmixing model includes ΔV(ω)=Σᵢ₌₁^{M} αᵢΔσᵢ(ω)+∈, where ΔV(ω) is the first, second, and third electric potential difference data sets, αᵢ is the parameter indicative of impact caused by tissue type i, Δσᵢ(ω) is predetermined spectrum specific to tissue type i, M is the total number of tissue types, ∈ is an error term. In one example the error term is 0, in which case the spectral unmixing model includes ΔV(ω)=Σᵢ₌₁^{M} αᵢΔσᵢ(ω), is the first and second electric potential difference data sets, αᵢ is the parameter indicative of impact caused by tissue type i, Δσᵢ(ω) is predetermined spectrum specific to tissue type i, M is the total number of tissue types. The system may include a memory that stores the spectral unmixing model and is operably connected with the one or more processors.

Optionally, the one or more processors are arranged to compare the determined value with a predetermined reference scale to determine whether the subject has a disease. The predetermined reference scale may include predetermined values of the parameter classified according to presence or absence of the disease, and optionally, severity of the disease.

Optionally, the one or more processors are arranged to process the electric potential difference data sets to provide a conductivity change map (e.g., average conductivity change map) of the subject.

Optionally, the parameter associated with the disease comprises a controlled attenuation parameter.

Optionally, the one or more anthropometric measures of the subject comprises a waist circumference over height (i.e., waist circumference of the subject divided by height of the subject) measure. Optionally, the one or more anthropometric measures of the subject comprises age of the subject. Optionally, the one or more anthropometric measures of the subject comprises chest circumference of the subject.

Optionally, the one or more processors are arranged to filter the electric potential data sets prior to determining the differences. The filtering may remove outlier(s).

Optionally, the disease comprises a liver disease, a lung disease, a kidney disease, etc. In one example, the disease comprises a fatty liver disease (e.g., nonalcoholic fatty liver disease).

Optionally, the one or more processors are arranged to determine, based on the processing, a severity of the disease.

Optionally, the system further comprises an output device, such as a display, arranged to present the determination result to the user. The presenting may include displaying the result to the user. The result may include a “yes/no” result (as to whether the subject has a disease) and optionally a severity of the disease.

Optionally, the subject is human being. Optionally the subject is a non-human animal.

In a seventh aspect, there is provided a non-transitory computer-readable medium storing computer instructions that, when executed by one or more processors, causes the one or more processors to perform the method of the fourth aspect.

In an eighth aspect, there is provided a computer program product comprising instructions which, when the program is executed by a computer, cause the computer to carry out the method of the fourth aspect.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

Terms of degree such that “generally”, “about”, “substantially”, or the like, are used, depending on context, to account for manufacture tolerance, degradation, trend, tendency, imperfect practical condition(s), etc. For example, when a value is modified by terms of degree, such as “about”, such expression may include the stated value ±10%, ±5%, ±2%, or ±1%.

Unless otherwise specified, the terms “connected”, “coupled”, “mounted”, and the like, are intended to encompass both direct and indirect connection, coupling, mounting, etc. Unless other specified, or context required otherwise, the term “conductivity”, and the like, means electrical conductivity or bio-conductivity.

### BRIEF DESCRIPTION OF THE DRAWINGS

Embodiments of the invention will now be described, by way of example, with reference to the accompanying drawings in which:

FIG. 1 is a flowchart illustrating a computer-implemented EIT data processing method in some embodiments of the invention.

FIG. 2 is a flowchart illustrating a computer-implemented EIT data processing method in some embodiments of the invention.

FIG. 3 is a schematic diagram illustrating a computer-implemented EIT data processing method in some embodiments of the invention.

FIG. 4 is a schematic diagram illustrating the steps used to predict CAP with frequency-difference EIT in one embodiment of the invention.

FIG. 5 is a plot showing decomposition of the total variance of the estimator into the variance due to repetition and the variance due to measurement noise with an area of reference for N_{R}=7 in one embodiment of the invention.

FIG. 6 is a plot of the VCTE estimated CAP and the predicted CAP using fd-EIT (blue) and fd-EIT with spectral unmixing (orange) method in one embodiment of the invention.

FIG. 7 are graphs showing average CAP values across healthy population (H) and non-healthy (NH), as classified by Fibroscan CAP: (A) the Fibroscan values, (B) EIT-based predicted CAP with classic fd-EIT, (C) EIT-based predicted CAP with unmixed polynomial.

FIG. 8 is a schematic diagram illustrating steps for data acquisition in one example.

FIG. 9 is a schematic diagram illustrating an example operation including EIT data acquisition, processing, and analysis in one embodiment of the invention.

FIG. 10 are graphs showing (A) scatter plot of CAP and predicted CAP, (B) average CAP values in different NAFLD stages classified by EIT-based predicted CAP, (C) average CAP values in different NAFLD stages classified by true CAP, (D) receiver operating characteristic (ROC) curve of predicted CAP for the healthy population against non-healthy population in one example.

FIG. 11 are graphs showing performance of Self-Assessment Score on (A) FibroScan CAP with (A1) scatterplots, (A2) average score classified by FibroScan CAP, and (A3) ROC curve for healthy vs non-healthy subjects in one example.

FIG. 12 are graphs showing (A) CAP Scatterplot of with stimulation data, (B) average CAP values in different NAFLD stages classified by EIT-based predicted CAP, (C) average CAP values in different NAFLD stages classified by true CAP, and (D) ROC curve of predicted CAP for healthy population against non-healthy population in one example.

FIG. 13A is a schematic diagram illustrating an example operation including EIT data acquisition, processing, and analysis in one embodiment of the invention.

FIG. 13B is a schematic diagram illustrating an example EIT data processing in one embodiment of the invention.

FIG. 14A is a graph showing linear correlation coefficients and relative importance of various EIT-features (conductivity characteristics) and anthropometric features of the subject in one example.

FIG. 14B is a graph (scatter plot) showing a relationship and classification specificity and sensitivity of an eGFR regression model (associated with true (blood test) eGFR value and EIT-determined eGFR score) in one embodiment of the invention.

FIG. 14C is a graph showing a receiver operating characteristic (ROC) curve of an eGFR regression model and classification scheme in one embodiment of the invention.

FIG. 14D are graphs showing EIT-determined eGFR score at different stages (S1-S5) and severities of chronic kidney disease in one example.

FIG. 15A is a graph showing simulation results obtained based on existing population eGFR distribution and error of the model in one embodiment of the invention.

FIG. 15B is a graph (scatter plot) showing simulated relationship and classification specificity and sensitivity of an eGFR regression model (associated with true (blood test) eGFR value and simulated EIT-determined eGFR score) in one embodiment of the invention.

FIG. 15C are graphs showing simulated EIT-determined eGFR score at different stages (S1-S5) and severities of chronic kidney disease in one example.

FIG. 16 is a block diagram of a data processing system arranged to perform one or more of the method embodiments (partly or entirely) in some embodiments of the invention.

FIG. 17 is a block diagram of an example EIT system in some embodiments of the invention.

FIG. 18 is a block diagram of an example EIT console in some embodiments of the invention.

FIG. 19 is a block diagram of an example EIT console in some embodiments of the invention.

### DETAILED DESCRIPTION

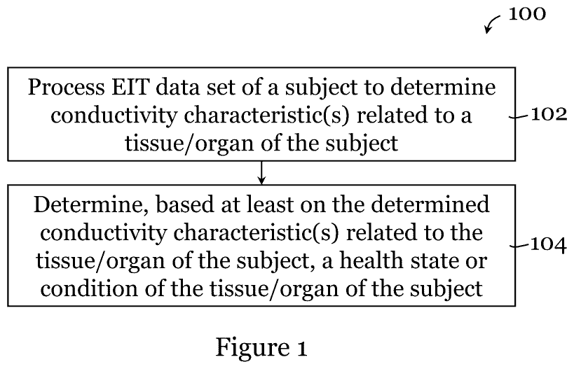

FIG. 1 shows a computer-implemented EIT data processing method 100 in some embodiments of the invention. The method includes, in step 102, processing a EIT data set of a subject to determine conductivity characteristic(s) related to a tissue/organ of the subject, and in step 104, determining a health state or condition of the tissue or organ of the subject based at least on the determined conductivity characteristic(s). In some embodiments, steps 102 and 104 are performed separately (sequentially). In some embodiments, steps 102 and 104 are performed at least partly simultaneously.

In some embodiments, the EIT data set contains EIT data obtain from a region (a body part) of the subject containing the organ or tissue of interest. The region may be a chest region, an abdominal region, etc. In some embodiments, the EIT data set is obtained by (a) providing excitation signals (voltage, potential, current, etc.) at a frequency to the subject via electrodes attached to the region of the subject, (b) measuring responsive signals (voltage, potential, current, etc.) received via the electrodes as a result of the providing of the excitation signals, and (c) repeating steps (a) and (b) for different frequencies. The EIT data set may comprise multiple EIT data subsets each associated with a respective one of the frequencies.

In some embodiments, step 102 may include one or more or all of:
(i) processing the EIT data set to obtain a processed EIT data set (which includes multiple processed EIT data subsets)
(ii) processing the processed EIT data set to obtain a frequency difference EIT data set (which includes multiple frequency difference EIT data subsets, the frequency difference EIT data subsets may correspond to multiple conductivity maps
(iii) performing a group source separation operation using the frequency difference EIT data set and one or more reference frequency difference EIT data sets of corresponding one or more reference subjects to determine component of the frequency difference EIT data set related to the tissue or organ of the subject and component of each of the one or more reference frequency difference EIT data sets related to the tissue or organ of the one or more reference subjects
(iv) performing a conductivity characteristics extraction operation using the component of the frequency difference EIT data set related to the tissue or organ of the subject and optionally the component of each of the one or more reference frequency difference EIT data sets related to the tissue or organ of the one or more reference subjects to determine at least the one or more conductivity characteristics of the subject
(v) performing a conductivity characteristics extraction operation using the processed EIT data set or the frequency difference EIT data set related to the tissue or organ of the subject and optionally each of the one or more processed EIT data sets or the one or more reference frequency difference EIT data sets related to the tissue or organ of the one or more reference subjects to determine at least one or more further conductivity characteristics

In some embodiments, (i) in step 102 may include filtering and/or smoothing each of the EIT data subsets.

In some embodiments, (i) in step 102 may additionally or alternatively include processing the EIT data set using a classifier model (e.g., a machine learning based processing model) to determine respective performance of each of the electrodes (the performance is associated with quality of responsive signals or data obtained from the respective electrode) when the EIT data is obtained and preventing the responsive signals or data obtained via any one or more of the electrodes determined to have insufficient performance from being included in the processed EIT data set. In some examples, a respective performance score is determined for each of the electrodes and the respective performance scores are compared with a reference data to determine whether the any of (and if so which) of the electrodes provided insufficient performance when the data is taken. In some examples, the classification model can determine the electrodes that have insufficient performance when the data is taken.

In some embodiments, (ii) in step 102 may include determining, for each respective one or more of the processed EIT data subsets, respective difference between the respective processed EIT data subset and a reference EIT data subset, so as to obtain the frequency difference EIT data subsets each associated with a respective one of a difference between the respective processed EIT data subset and a reference EIT data subset. At least one of the processed EIT data subsets may be respectively used as the reference EIT data subset.

In some embodiments, (iii) in step 102 may include performing a dimensionality reduction operation on the frequency difference EIT data set and one or more reference frequency difference EIT data sets of corresponding one or more reference subjects to determine component of the frequency difference EIT data set related to the tissue or organ of the subject and respective component of each of the one or more reference frequency difference EIT data sets related to the tissue or organ of the one or more reference subjects.

In some embodiments, (iv) in step 102 may include determining, using the component of the frequency difference EIT data set related to the tissue or organ of the subject, the one or more (e.g., statistical) conductivity characteristics related to the tissue or organ of the subject. For example, the one or more (e.g., statistical) conductivity characteristics related to the tissue or organ of the subject may include one or more of: an average of conductivity characteristics in a defined tissue or organ region within an area, an average of conductivity characteristics outside the defined tissue or organ region within the area, and an average of conductivity characteristics within the area. The area may be an area within the conductivity map. In some embodiments, other statistical conductivity characteristics related to the tissue or organ of the subject, such as median, mode, standard deviation, etc., may be used instead of the mean.

In some embodiments, (iv) in step 102 may include determining, using the component of the frequency difference EIT data set related to the tissue or organ of the subject and respective component of each of the one or more reference frequency difference EIT data sets related to the tissue or organ of the one or more reference subjects, one or more (e.g., statistical) conductivity characteristics related to the tissue or organ of a group containing the subject and the one or more reference subjects. And, the determining of the health state or condition of the tissue or organ of the subject may be further based on the one or more conductivity characteristics related to the tissue or organ of the group. For example, the one or more (e.g., statistical) conductivity characteristics related to the tissue or organ of the group may include one or more of: an average of conductivity characteristics in a defined tissue or organ region within an area, an average of conductivity characteristics outside the defined tissue or organ region within the area, and an average of conductivity characteristics both in and outside the defined tissue or organ region within the area. The area may be an area within the conductivity map (e.g., averaged conductivity map determined from the conductivity maps). In some embodiments, other statistical conductivity characteristics related to the tissue or organ, such as median, mode, standard deviation, etc., may be used instead of the mean.

In some embodiments, step 104 includes: determining, based on at least the one or more determined conductivity characteristics, whether the subject has a disease associated with the tissue or organ. In some embodiments, step 104 also includes classifying a stage or a severity of the disease associated with the tissue or organ.

In some embodiments, step 104 includes: determining, based on at least the one or more determined conductivity characteristics, a value associated with an estimated performance of the tissue or organ of the subject. For example, in respect of kidney, the value may be an estimated glomerular filtration rate or a related value (e.g., arithmetically related). For example, in respect of liver, the value may be a controlled attenuation parameter (CAP) score or a related value (e.g., arithmetically related).

In some embodiments, step 104 includes: processing, at least, the one or more determined conductivity characteristics of the subject and one or more anthropometric characteristics of the subject, using a machine learning based processing model, to determine a quantitative or qualitative parameter associated with the health state or condition of the tissue or organ of the subject.

In some embodiments, step 104 includes: processing, using a machine learning based processing model, (i) the one or more determined conductivity characteristics of the subject, (ii) one or more anthropometric characteristics of the subject, and (iii) one or more determined conductivity characteristics of one or more reference subjects and/or one or more determined conductivity characteristics of a group containing the subject and the one or more reference subjects, to determine a quantitative or qualitative parameter associated with the health state or condition of the tissue or organ of the subject. Preferably, the one or more reference subjects do not suffer from any disease associated with the tissue or organ.

The machine learning based processing model may include a regression model, a classification model, etc. The regression model may include a linear regression model or a non-linear regression model.

The one or more anthropometric characteristics may include or be related to one or more of, e.g.: age of the subject, weight of the subject, height of the subject, and waist circumference of the subject, waist-over-height ratio of the subject, body mass index (BMI) of the subject, gender of the subject, and race of the subject.

The quantitative or qualitative parameter associated with the health state or condition of the tissue or organ of the subject may include a value associated with an estimated performance of the tissue or organ of the subject. For example, in respect of kidney, the value may be an estimated glomerular filtration rate or a related value (e.g., arithmetically related). For example, in respect of liver, the value may be a controlled attenuation parameter (CAP) score or a related value (e.g., arithmetically related).

In some embodiments, step 104 includes: comparing the quantitative or qualitative parameter associated with the health state or condition of the tissue or organ of the subject with reference parameter data (reference parameter value(s) or range(s)) to determine whether the subject has a disease associated with the tissue or organ. In some embodiments, step 104 further includes: classifying, based on the comparing, a stage or a severity of the disease associated with the tissue or organ.

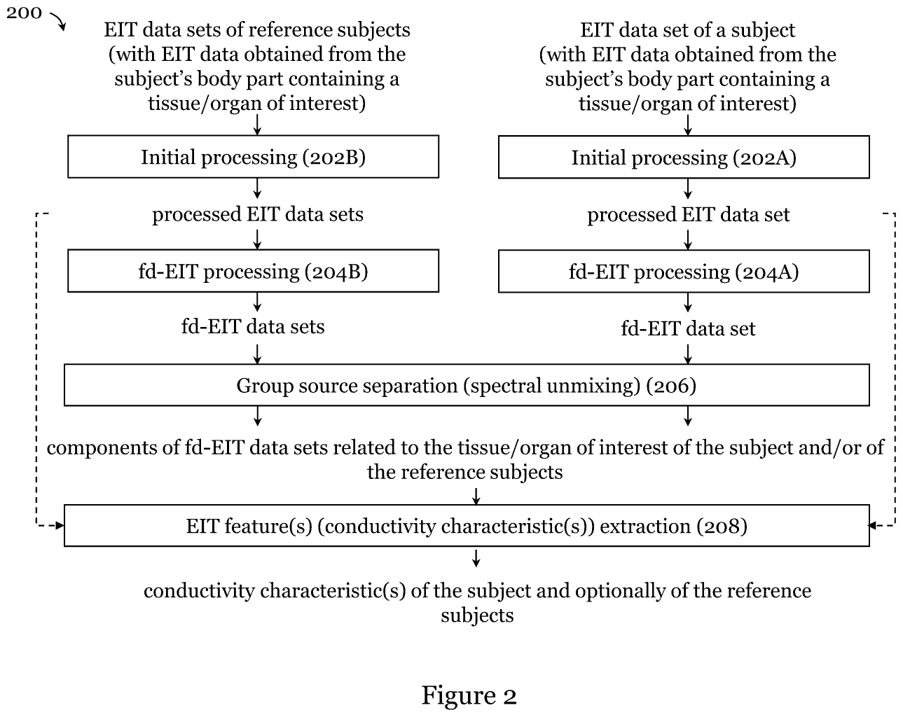

FIG. 2 shows a computer-implemented EIT data processing method 200 in some embodiments of the invention.

The method 200 includes, in step 202A, performing an initial processing (pre-processing) on a EIT data set of a subject to obtain a processed EIT data set. The EIT data set includes EIT data obtained from the subject's region containing a tissue or organ of interest. The EIT data set may be obtained using the method 100 described above with reference to FIG. 1. The initial processing may include the processing mentioned with respect to step 102, (i), in the method 100 described above with reference to FIG. 1.

The method 200 includes, in step 204A, performing a fd-EIT processing operation on the processed EIT data set to obtain a fd-EIT data set of the subject. The fd-EIT processing operation may include the processing mentioned with respect to step 102, (ii), in the method 100 described above with reference to FIG. 1. The fd-EIT processing operation may be other fd-EIT method known in the art to obtain conductivity maps.

The method 200 may also include, in step 202B, performing an initial processing (pre-processing) on EIT data sets of reference subjects (who do not has any disease associated with the tissue or organ of interest) to obtain a processed EIT data set. The EIT data sets each includes EIT data obtained from a respective reference subject's region containing the tissue or organ of interest. The EIT data sets may be obtained using the method 100 described above with reference to FIG. 1. The initial processing may include the processing mentioned with respect to step 102, (i), in the method 100 described above with reference to FIG. 1.

The method 200 may also include, in step 204B, performing a fd-EIT processing operation on the processed EIT data sets to obtain fd-EIT data sets of the reference subjects. The fd-EIT processing operation may include the processing mentioned with respect to step 102, (ii), in the method 100 described above with reference to FIG. 1. The fd-EIT processing operation may be other fd-EIT method known in the art to obtain conductivity maps.

The method 200 includes, in step 206, performing a group source separation operation using the fd-EIT data set of the subject and fd-EIT data sets of reference subjects. The main aim of the group source separation operation is to separate or extract the source signals (i.e., the signals or data related to the tissue or organ of interest) from the fd-EIT data set of the subject and fd-EIT data sets of reference subjects, which may include signals of other organs or tissues. The result of step 206 is the obtaining of components of fd-EIT data sets related to the tissue or organ of the reference subjects and component of fd-EIT data set related to the tissue or organ of the subject. In one example, if the one fd-EIT data set of the subject and two fd-EIT data sets of reference subjects, then after the group source separation operation, one set of data (a map) containing component of fd-EIT data set related to the tissue or organ of the subject and two sets of data (two maps) containing components of fd-EIT data set related to the tissue or organ of the reference subjects will be obtained. The group source separation operation may include the processing mentioned with respect to step 102, (iii), in the method 100 described above with reference to FIG. 1.

The method 200 includes, in step 208, performing EIT feature(s) extraction operation using the components of fd-EIT data sets related to the tissue or organ of the reference subjects and component of fd-EIT data set related to the tissue or organ of the subject. Here, the EIT feature(s) correspond to the conductivity characteristic(s) or feature(s). The result of step 206 is the obtaining of feature(s) (conductivity characteristic(s)) of the subject and optionally of the reference subjects. The EIT feature(s) extraction operation may include the processing mentioned with respect to step 102, (iv) and (v), in the method 100 described above with reference to FIG. 1. In one example, if there exists one set of data (a map) containing component of fd-EIT data set related to the tissue or organ of the subject and two sets of data (two maps) containing components of fd-EIT data set related to the tissue or organ of two reference subjects, then one or more of these maps will be processed to determine the feature(s) related to the tissue or organ. For example, a statistical operation may be performed on the entire map of containing component of fd-EIT data set related to the tissue or organ of the subject to obtain an average, mean, median, etc., of conductivity of the map.

For example, a statistical operation may be performed on only part of the map of containing component of fd-EIT data set related to the tissue or organ of the subject to obtain an average, mean, median, etc., of conductivity of that part of the map. For example, a statistical operation may be performed on all of the maps containing components of fd-EIT data set related to the tissue or organ of the subject and the reference subjects to obtain an average, mean, median, etc., of conductivity of the aggregate of maps. For example, a delineation operation (e.g., thresholding) may be performed on the map of containing component of fd-EIT data set related to the tissue or organ of the subject to define a region of interest and obtain an average, mean, median, etc., of conductivity of that region of interest of the map. These can all be obtained as the EIT feature(s). In some examples, the processed EIT data set of the subject and/or the processed EIT data sets of the reference subjects can also be used in the performing of the EIT feature(s) extraction operation.

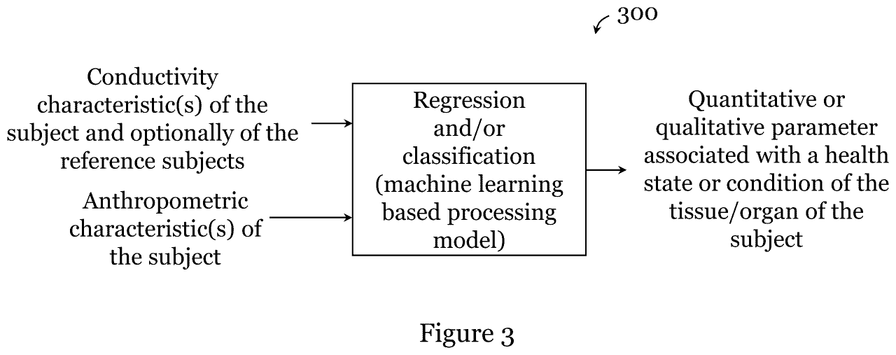

FIG. 3 shows a computer-implemented EIT data processing method 300 in some embodiments of the invention. The method 300 includes processing the conductivity characteristic(s) associated with the subject and optionally the reference subjects and the anthropometric characteristic(s) of the subject using a regression and/or classification model (e.g., machine learning based processing model) to determine a quantitative or qualitative parameter associated with a health state or condition of the tissue or organ of the subject. The EIT feature(s) can be those in the methods 100, 200 described above with reference to FIGS. 1 and 2. The anthropometric characteristic(s) can be those in the method 100 described above with reference to FIG. 1.

The following provide some example embodiments of the invention. These embodiments may be considered as specific example implementations of the method 100 in FIG. 1.

### Example 1

Example 1 can be considered as a specific example implementation of the method 100 of FIG. 1 with the tissue/organ of interest being the liver.

### 1. Introduction

### 1.1 Fatty Liver, Controlled Attenuation Parameter, and Aim of the Embodiment

Nonalcoholic fatty liver disease (NAFLD), also known as hepatic steatosis, is the apparition of fat around hepatocytes (liver cells). NAFLD is typically associated with sedentary lifestyle.

Currently various techniques are used to diagnose NAFLD. One technique is liver biopsy. This technique, while useful, is invasive, relatively expensive, prone to sampling error, often painful, and might result in some severe complications. Non-invasive techniques based on ultrasound-based devices and vibration-controlled transient elastography are also used to diagnose NAFLD. These techniques measure liver elasticity to infer hepatic steatosis in NAFLD quantified using the controlled attenuation parameter.

In this embodiment, frequency sweeping (i.e., multiple measurements each at a different frequency) in EIT is applied to predict controlled attenuation parameter from cross-sectional EIT measurement across the liver, with both the classic frequency difference and the spectral unmixing model.

### 1.2 General Principle of Electrical Impedance Tomography

Electrical impedance tomography (EIT) is a noninvasive imaging method based on measuring electrical impedance of living tissue (i.e., bio-impedance). Typically a small electrical current (e.g., about 1 mA, or any other value, which does not affect normal physiology) is applied into the body through electrodes (e.g., on a belt) at frequencies ranging typically from 1 kHz to 1 MHz (other frequencies are also possible). This electrical current induces an electric potential that is measured at each electrode. Using this input, a map of the conductivity inside the body is reconstructed. These changes in conductivity are used to predict controlled attenuation parameter values. In the remainder of this section, we briefly describe the EIT reconstruction problem.

### 1) Forward Problem

Assume that a current is applied to the body (of a subject) through a source electrode and a responsive signal (e.g., electric potential) is received at a sink electrode. If the conductivity inside the body is known, it would be possible to compute the electric potential inside the body using Maxwell's equations

$$\{ \begin{matrix}∇ \cdot ( σ ∇ V ) = 0 inside the body \\ \int σ \frac{\partial V}{\partial n} = \pm I at the \frac{source}{sink} electrode \\ \frac{\partial V}{\partial n} = 0 at the other boundaries\end{matrix}$$

where σ denotes the conductivity inside the body and V the electric potential. This equation can be simulated quite accurately.

### 2) Inverse Problem

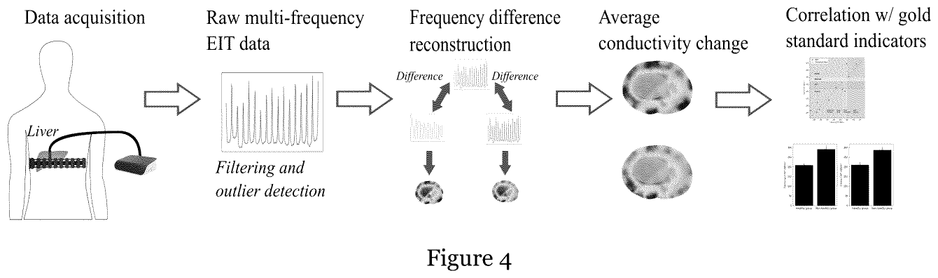

The main challenge of EIT is that neither the conductivity a nor the potential V inside the body is known. Instead, they must be recovered from boundary measurements Vₘₑₛ, and this leads to an inverse problem. This example aims to recover only the change in conductivity between two frequencies (FIG. 4). Using linear approximation, this type of reconstruction is easier than absolute imaging. The algorithm used in this embodiment is the linearized least-square algorithm with Kotre regularization and prior information extracted from CT scans. By denoting the forward operator that maps the conductivity to the measured potential using Vₘₑₛ=F(σ), then the change in potential can be expressed as follows:

$${V}_{mes} - {V}_{mes , 0} = F ( σ ) - F ( {σ}_{0} ) ≃ ∇ F ( {σ}_{0} ) \cdot ( σ - {σ}_{0} )$$

This relationship is not enough to recover a from boundary measurement, because of the ill-posedness of the problem. A regularization term and a prior information are incorporated, so that the discretized final expression of the conductivity is

$$( σ - {σ}_{0} ) = {( ∇ {F}^{T} ∇ F + λ ( ( 1 - α ) {R}^{p} + α {W}_{x} ) )}^{- 1} \cdot ∇ {F}^{T} \cdot ( {V}_{mes} - {V}_{mes , 0} )$$

where R is the Kotre diagonal sensitivity matrix, α and p two regularization parameter and Wₓ a matrix that incorporates prior information.

### 2. Material and Methods

### 2.1 Clinical Assessment

Tests are performed on a total of 11 human subjects including 3 females and 8 males, from 20 to 65 years old, with a waist circumference from 71 cm to 110 cm. Individual clinical demographics and physical characteristics, including gender, BMI, age, waist circumference, height, weight, and liver disease history (if any), are collected.

### 2.2 Data Acquisition Process

Each of the subjects is first tested for 5-10 minutes liver FibroScan® session (Echosens, France) to obtain the controlled attenuation parameter (CAP) value. EIT examination is then performed with a portable system with a EIT data acquisition console and a 16-electrode belt. The data acquisition console used in this example consists of a power management module, with a current generator providing alternating current at frequencies ranging from 10 KHz to 1 MHz (other frequency ranges, e.g., in the order of GHz, are also envisaged), a data acquisition module for potential difference measurement, and a control and output module for module coordination, data processing and cloud-server communication. The position of the electrode belt (hence the electrodes on it) targets the upper abdominal region, as indicated by the bottom boundary of the ribcage.

### 2.3 Classic Frequency-Difference

The conductivity is reconstructed using a custom version of the pyEIT python library. The changes in conductivity between two pairs of frequency (28.75 kHz-20.00 kHz) and (28.75 kHz-25.00 kHz) are computed by averaging the conductivity map of the difference over the region of interest (ROI) covering the whole liver. Both the prior and region of interest are extracted from a CT scan image.

### 2.4 Spectral Unmixing Method for Conductivity

In this example, a method to extract more information from the frequency-difference curve is provided. This method is based on that if the change in conductivity with respect to frequency is small, then the shape of the measured voltage over frequency is given by a linear combination of the shape of the conductivity changes over frequency. For example, approximate the conductivity change over frequency by its Taylor expansion:

$$σ ( x , ω ) - σ ( x , {ω}_{0} ) = \frac{\partial σ ( x , {ω}_{0} )}{\partial ω} ( ω - {ω}_{0} ) + \frac{{\partial }^{2} σ ( x , {ω}_{0} )}{\partial {ω}^{2}} \frac{{( ω - {ω}_{0} )}^{2}}{2} + O ( {( ω - {ω}_{0} )}^{2} ) .$$

The measured potential is equal to Vₘₑₛ(ω)=F(σ(ω)), which is approximated by the discrete operator Vₘₑₛ(ω)≈\tilde{F}(σ(x_{N},ω)), where x_{N} is vector of points that discretizes the domain. The difference of potential can be approximated as follows

$${V}_{mes} ( ω ) - {V}_{mes} ( {ω}_{0} ) = ( ∇ \tilde{F} ( ( σ ( {x}_{N} , {ω}_{0} ) ) ) \cdot \frac{\partial σ ( {x}_{N} , {ω}_{0} )}{\partial ω} ) ( ω - {ω}_{0} ) + ( ∇ \tilde{F} ( ( σ ( {x}_{N} , {ω}_{0} ) ) ) \cdot \frac{{\partial }^{2} σ ( {x}_{N} , {ω}_{0} )}{\partial {ω}^{2}} ) \frac{{( ω - {ω}_{0} )}^{2}}{2} + o ( {( ω - {ω}_{0} )}^{2} ) + o ( ‖ σ ( {x}_{N} , ω ) - σ ( {x}_{N} , {ω}_{0} ) ‖ ) \qquad ( 2 )$$

and the derivatives can be identified as

$$\begin{matrix}\frac{\partial V}{\partial ω} ( {ω}_{0} ) ≃ ∇ \tilde{F} ( {( σ ( {x}_{k} , {ω}_{0} ) )}_{k ∈ \{ 1 , … , N \}} ) \cdot {( \frac{\partial σ ( {x}_{k} , {ω}_{0} )}{\partial ω} )}_{k ∈ \{ 1 , … N \}} \\ \frac{{\partial }^{2} V}{\partial {ω}^{2}} ( {ω}_{0} ) ≃ ∇ \tilde{F} ( {( σ ( {x}_{k} , {ω}_{0} ) )}_{k ∈ \{ 1 , … , N \}} ) \cdot {( \frac{{\partial }^{2} σ ( {x}_{k} , {ω}_{0} )}{\partial {ω}^{2}} )}_{k ∈ \{ 1 , … N \}}\end{matrix}$$

It should be noted that the relationship between

$$\frac{\partial V}{\partial ω} ( {ω}_{0} ) ( resp . \frac{{\partial }^{2} V}{\partial {ω}^{2}} ( {ω}_{0} ) ) and \frac{\partial σ ( {x}_{k} , {ω}_{0} )}{\partial ω} ( resp . \frac{{\partial }^{2} σ ( {x}_{k} , {ω}_{0} )}{\partial {ω}^{2}} )$$

is the same as the relationship between ΔV and Δσ. Thus, the exact same methods used to reconstruct Δσ can be used to reconstruct

$$\frac{\partial σ ( {x}_{k} , {ω}_{0} )}{\partial ω} ( resp . \frac{{\partial }^{2} σ ( {x}_{k} , {ω}_{0} )}{\partial {ω}^{2}} ) .$$

It is assumed that the parts of the body that cause first order and second order change are different, and thus isolating them would provide more specific information. Note that this method example has been presented with an expansion up to two orders. However, it can be extended to higher orders, provided the conductivity changes have enough derivatives. It can also be extended to any functional basis/free family decomposition. Assume that M tissue types are contributing to the conductivity change and they each have a conductivity variation of Δσᵢ(ω), then, under the linearity assumption (2), the following can be obtained:

$$Δ V ( ω ) = {\sum }_{i = 1}^{M} {a}_{i} {Δσ}_{i} ( ω ) + ϵ$$

where ΔV(ω) is the electric potential difference data sets (except the reference set), αᵢ is the parameter indicative of impact caused by tissue type i, Δσᵢ(ω) is predetermined spectrum specific to tissue type i, M is the total number of tissue types, ∈ is an error term. The Δσᵢ(ω) can be obtained by tabulated values and the coefficient can be estimated by classic linear mixed model methods (Table I).

TABLE I
Summary of the recovered map with respect to the given inputs

| | | | | |
|---|---|---|---|---|
| Input | ΔV | $\frac{\partial V}{\partial ω}$ | $\frac{{\partial }^{2} V}{\partial {ω}^{2}}$ | αᵢ (coefficient due to organ i) |
| Recovered map | Δσ | $\frac{\partial σ}{\partial ω}$ | $\frac{{\partial }^{2} σ}{\partial {ω}^{2}}$ | Δσᵢ (change at organ i) |

### 2.5 Optimization of the Acquisition Paradigm

Once the model for the controlled attenuation parameter is constructed, more EIT data is acquired (without measuring the vibration-controlled transient elastography controlled attenuation parameter) to minimize the variance of the prediction. For each frequency acquisition, the duration of the acquisition and the number of repetitions can be freely chosen. Assume that the data acquired at the frame i for the repetition j provides an observation of the controlled attenuation parameter of

$${\overline{C A P}}_{i j} = C A P + {ε}_{F , i , j} + {ε}_{R j}$$

where CAP is the true value of the controlled attenuation parameter, ∈_{F,i,j} is the measurement error (i.e., due to sensor noise) and ∈_{R,j} is the repetition error (the patient does not repeat exactly the same pattern). Denote by N_{F} the number of frames acquired per repetition and by N_{R} the number of repetitions; to estimate the controlled attenuation parameter, the Monte-Carlo average is used over all the frames

$${i , j}_{} = CAP + {ϵ}_{F , i , j} + {ϵ}_{R , j}$$

Assuming that the ∈_{F,i,j}, ∈_{R,j} are independent random variables and the errors ∈_{F,i,j} (resp. ∈_{R,j}) share the same finite variance σ_{F}² (resp. σ_{R}²), by the central limit theorem, the following can be obtained:

$$Var ( \overline{CAP} ) ≃ \frac{{σ}_{F}^{2}}{{N}_{F} {N}_{R}} + \frac{{σ}_{R}^{2}}{{N}_{R}} ,$$

when N_{R} and N_{F} are large enough. In this example duration of one acquisition is modelled by the product of the number of frames N_{F} and the duration of the acquisition of one frame t_{F} plus a setup time t_{S}. The total duration of all the acquisitions is then T=N_{R} (N_{F}t_{F}+t_{S}).

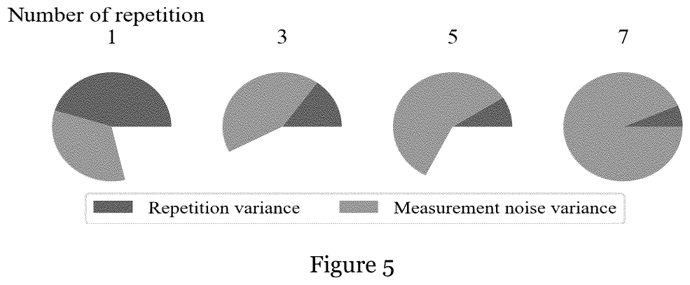

By fixing the total time T=T₀ and the previous equations, the total variance can be decomposed into the part due to measurement noise and the part due to repetition (FIG. 5). The best theoretical value can be computed, and are

$${N}_{F} = \sqrt{\frac{{t}_{S}}{{t}_{F}}} \frac{{σ}_{F}}{{σ}_{R}} and {N}_{R} = \frac{{T}_{0}}{\frac{\sqrt{{t}_{S} {t}_{F}} {σ}_{F}}{{σ}_{R}} + {t}_{S}} .$$

Finally, the values are chosen by rounding off the obtained real values. For a setup time of t_{S}=5 s and a total acquisition time of T₀=30 s per frequency, the optimal number of repetitions is N_{R}=3 and the acquisition time is N_{F}t_{F}=5 second.

### 3. Results

3.1 Linear Regression with Simple Difference

TABLE II
Results of the linear regression of the controlled attenuation
parameter vs change in conductivities and WoH.

| | | | |
|---|---|---|---|
| Dep. Variable: | controlled | R² | 0.935 |
| | attenuation | | |
| | parameter | | |
| F-statistic: | 57.42 | Adjusted-R² | 0.919 |
| Features (normalized by standard | Coefficient | Standard error | P > \|t\| |
| deviation) | | | |
| dC_{28-22 KHz} | −18.4983 | 6.119 | 0.016 |
| dC_{28-25 KHz} | −19.6607 | 6.248 | 0.014 |
| WoH | 33.4490 | 1.393 | 0.000 |
| Mean Average Percentage Error | 6% | No. of Observations | 11 |
| (Leave One Out) | | | |

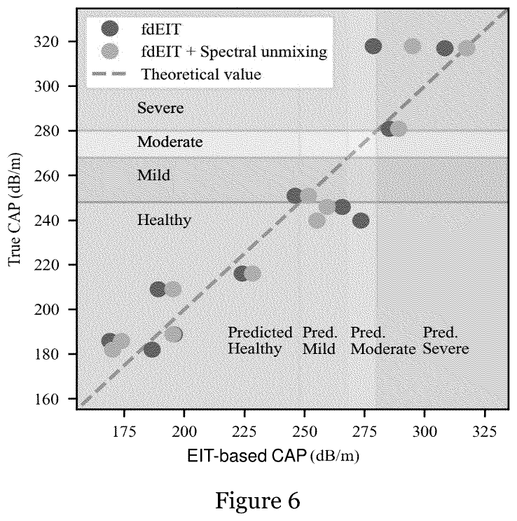

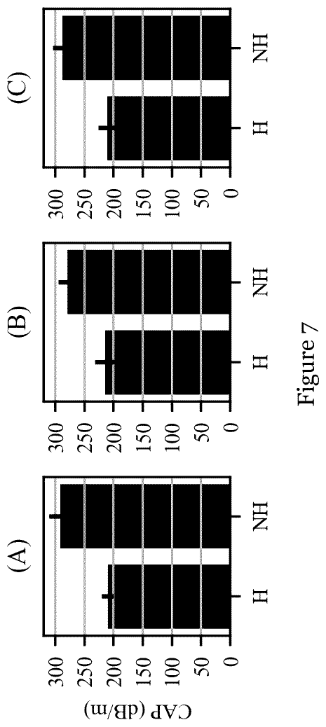

In this example, the controlled attenuation parameter is predicted by a linear regression using as features the spatial average of the change in conductivity dC_{28.75kHz-22kHz}, dC_{28.75kHz-25kHz} and the anthropometric variable WoH (waist circumference over height). All the variables are significant under a student t-test at level 0.05%. The coefficient of determination is 0.932 and the adjusted one is 0.919 (Table II). The predicted controlled attenuation parameter seems to be able to classify the liver state based on the FibroScan controlled attenuation parameter, (sensitivity=0.75 and a specificity=0.71, the subject being incorrectly classified being mostly close to the threshold, FIGS. 6 and 7). The regression keeps a good homoscedasticity, even for high value of the controlled attenuation parameter.

From Table III, it can be seen that including both conductivity and WoH improves greatly the quality of the regression, compared to using solely one or the other. The coefficients of the linear regression using normalized data being of the same order of magnitude, it can be determined that they have the same importance in the prediction.

TABLE III
Coefficient of determination for different models

| Features used in the | | dC_{28-22 KHz} & | dC_{28-22 KHz} & |
|---|---|---|---|
| linear regression | WoH | dC_{28-25 KHz}& intercept | dC_{28-25 KHz} & WoH |
| R² | 0.425 | 0.444 | 0.935 |
| R²-adjusted | 0.425 | 0.305 | 0.919 |

3.2 Linear Regression with Unmixed Polynomial Coefficients

As explained above, the first order and second order variation of V with respect to ω are used to reconstruct the conductivity. For each patient or subject the α and β are estimated using least-squares verifying Vₘₑₛ (ω)−Vₘₑₛ(ω₀)≈α(ω−ω₀)+β(ω−ω₀)² and are then used to reconstruct an image. The average conductivity in the liver area dC_{α} and dC_{β} are computer and used as regressors. This polynomial model provides a good approximation of the change in potential between 10 kHz and 35 kHz (MAPE of 4%). Their performances in the predicted cap are similar to the simple difference, with an adjusted R-squared of 0.914 with WoH (FIGS. 6 and 7).

### 4. Discussion

### 4.1 Controlled Attenuation Parameter Prediction

The data in this example suggests a strong correlation between the difference of conductivity across two frequency pairs and the controlled attenuation parameter measured by vibration-controlled transient elastography, in addition to the already observed correlation with the waist circumference over height (WoH). This correlation can be explained by the conductivity change with respect to the fat content in liver tissue, and is captured by EIT. A shape prior is used to focus on the liver region. Furthermore, the use of a self-administrable EIT device instead of a vibration-controlled transient elastography permits to have a more affordable measure with a real-time result, without requiring the help of a trained professional for acquisition.

### 4.2 Spectral Unmixing Model

The spectral unmixing method in one embodiment provides results similar to classic fd-EIT (adj. R-squared of 0.914), confirming the validity of the approach. Due to its reasonable assumptions, this approach can be applied to other organs (e.g. kidney).

In summary, this embodiment shows that multi-spectral electrical impedance tomography can predict clinical-standard controlled attenuation parameter in patients with or without nonalcoholic fatty liver disease using waist over height (WoH) anthropometric as complementary information. This embodiment also provides a spectral unmixing method to estimate controlled attenuation parameter from multi-spectral EIT by matching the coefficient of a functional decomposition. This spectral unmixing method can be applied for processing other EIT data for diagnosing different diseases (other than liver disease). This embodiment also provides an algorithm to determine the optimal number of repetitions (N_{R}=3) and acquisition time (N_{F}t_{S}=5 s) that minimizes the error and maximizes the accuracy under a constraint of time. In one example, these aspects are all independent from each other (i.e., implemented separately). In another example, two or more of these aspects are implemented at the same time.

### Example 2

Example 2 can be considered as a specific example implementation of the methods 100-300 in FIGS. 1-3 with the tissue/organ of interest being the liver.

### 1. Introduction

This example relates to EIT based systems and methods for assessing health state or condition of the liver of a subject (e.g., whether the subject has non-alcoholic fatty liver disease (NAFLD)).

### 2. Material and Methods

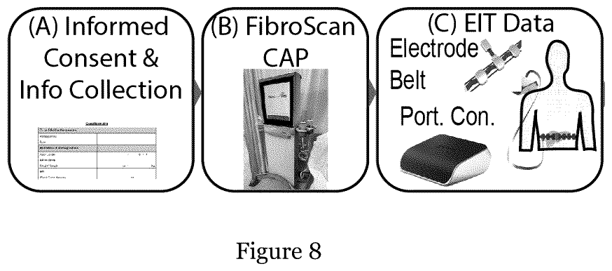

FIG. 8 shows steps for the acquisition of data in the example (“Port. Con.” means portable console).

### 2.1 Data Acquisition

64 human subjects are included in the experiment of this example. These subjects range from 21 years old to 84 years old, with waist circumferences from 65 cm to 127 cm and BMI from 18.9 to 43.3. The subject's body measure information including gender, BMI, age, waist circumference, height and prior medical history are collected (FIG. 8). CAP is then acquired with FibroScan (Echosens, France) followed by a 3-time EIT examination with a 16-electrode belt worn on the waist and portable system. The current injected ranges from 10 KHz to 400 KHz.

### 2.2 Constant Reference EIT, Frequency-Difference EIT and Group Source Separation

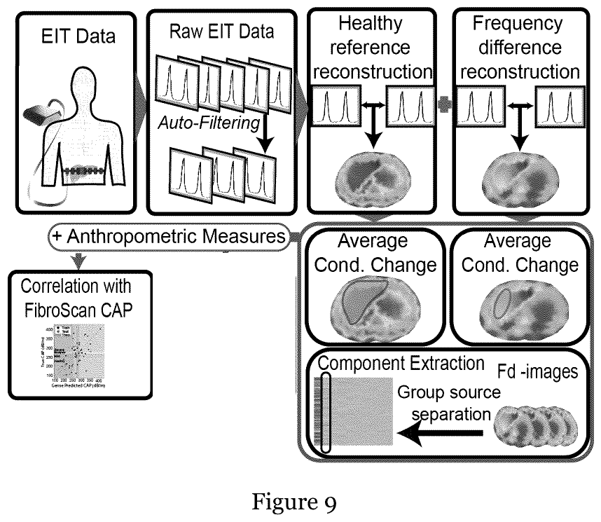

FIG. 9 shows the steps involved in the generation of features for the prediction of FibroScan CAP in this example (“F-d” means frequency-difference).

Data are first automatically filtered based on EIT quality (FIG. 9). Both constant reference conductivity images and frequency-difference EIT images are generated with a modified version of the pyEIT library. For constant reference EIT, the average of healthy subjects' conductivity map classified by CAP at 50 kHz frequency is chosen as reference. Conductivity difference EIT data between 160 kHz and 80 kHz are chosen as another feature. The prior and ROI within the whole liver are extracted and modified from a liver CT scan image. In this example the group source separation utilizes every possible pair of frequency-difference data between 20 kHz and 160 kHz. The dataset is split into train/test sets to generalize the separation model to unmix source components.

### 2.3 Regression Models

In this example the CAP prediction (CAP prediction using EIT data) model is a linear regression model with the above 3 features, and subject's waist-over-height ratio (WtHt), height, weight, age, and gender with train/development/test split. The model is built using the scikit-learn library in python in this example.

### 3. Results

### 3.1 Regression Model Performance on CAP

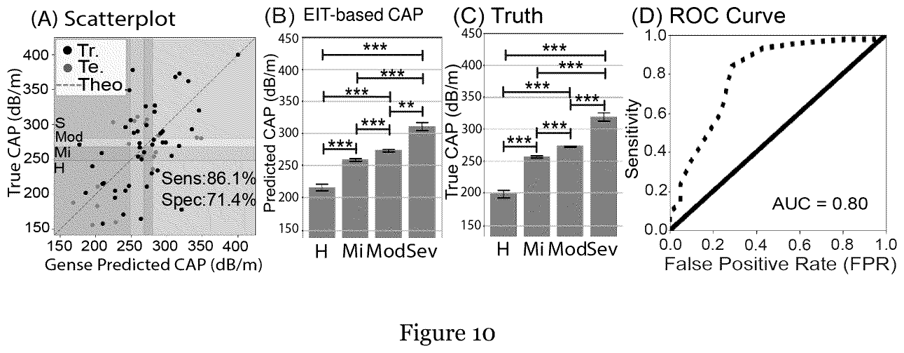

FIG. 10 shows (A) scatter plot of CAP and EIT-based predicted CAP. Average CAP values in different NAFLD stages classified by (B) EIT-based predicted CAP, (C) CAP. (D) Receiver Operating Characteristic (ROC) curve of EIT-based predicted CAP for the healthy population against non-healthy population. (“Theo” means theoretical value; “Tr” means training, “Te” means testing, “Theo.” means theoretical, “H” means Healthy, “Mi” means mild, “Mod” means moderate, “Severe” means severe, ***: p<0.001***: p<0.01).

The scatterplot of all the (EIT-based) predicted CAP against true CAP values is shown in FIG. 10. The (EIT-based) predicted CAP values have a sensitivity of 86.1% and a specificity of 71.4% (coefficient of determination=0.25). One-way ANOVA and posthoc Bonferroni test between each stage of NAFLD indicate that all progressive NAFLD stages predicted by both CAP is statistically different from other groups (p<0.001). The (EIT-based) predicted CAP achieved 0.799 in the area under curve (AUC) of the receiver operating characteristics (ROC) curve.

### 4. Discussion

### 4.1 Model Feature Selection

The 3 features used in this example uncover more information about the status of the liver. In this example, for constant reference EIT, 50 kHz is chosen as the target frequency while conventional time-difference EIT is modified with the reference constant reference EIT. As the mean of all available healthy classified by CAP is chosen as the reference conductivity data, the unique characteristics of each subject can be captured in the feature.

For group source separation, grouping all the combinations of fd-EIT ideally maximize the signal difference between fat and liver tissue, thus highlighted properties can be unmixed to produce a feature correlating to fat tissue.

For fd-EIT, the frequency 160 kHz and the reference frequency 80 kHz are chosen.

### 4.2 Clinical Significance

To demonstrate clinical screening performance, a self-assessment score disclosed in Y.-ho Lee, et al., PLoS ONE, vol. 9, no. 9, 2014 is compared with the EIT-based predicted CAP.

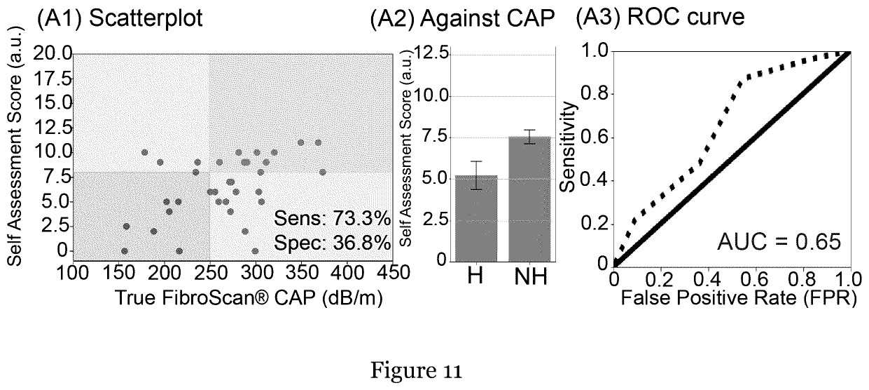

FIG. 11 shows the results, i.e., the performance of Self-Assessment Score on (A) FibroScan CAP with (1) scatterplots, (2) average score classified by FibroScan CAP, and (3) ROC curve for healthy vs non-healthy subjects.

As shown in FIG. 11, the self-assessment score suffers bias in their prediction, with a sensitivity of 73.3% and specificity of 36.8% compared to CAP. Contrary to the predicted values of the method of this example, the assessment scores do not produce statistically different results between healthy and non-healthy groups. The AUC of ROC values is 0.654 classified by CAP, lower than our predicted values of AUC 0.79.

To predict the model's performance in this example, additional data points are simulated based on the variance of existing model to match the population distribution from the National Health and Nutrition Examination Survey Data (NHANES) database using ADAPT library.

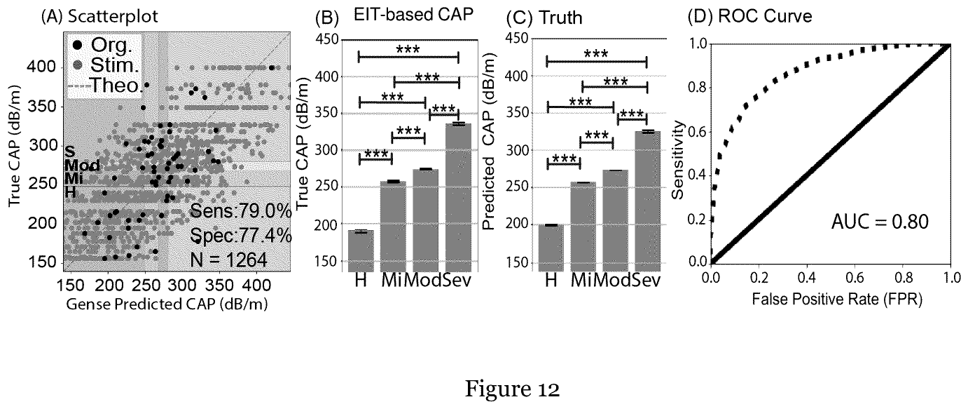

FIG. 12 shows the related results (A) CAP Scatterplot of with stimulation data. Average CAP values in different NAFLD stages classified by (B) EIT-based predicted CAP and (C) true CAP. (D) ROC curve of Predicted CAP for healthy population against non-healthy population (“Org” means original data points, “Stim” means stimulated data points, “Theo” means theoretical data points, ***: p<0.001)

It is predicted that sensitivity and specificity stay at around 77% while the AUC of ROC is maintained at 0.80(N=1264), as shown in FIG. 12. The performance of the model of this example is maintained with a larger dataset—this demonstrates the potential of the method and system of this example as a low-cost portable tool for home- and community-based NAFLD screening and monitoring.

Overall, this example demonstrates that EIT can predict clinical standard VCTE FibroScan CAP with anthropometric measures and conductivity features. This example has also demonstrated a cost-effective and self-administrable alternative for home- and community-based NAFLD widespread diagnostic screening and monitoring.

### Example 2

Example 3 can be considered as a specific example implementation of the methods 100-300 in FIGS. 1-3 with the tissue/organ of interest being the kidney(s).

In this example: a data processing pipeline to extract the kidney signals from in vivo EIT data is provided, and a regression model to estimate the eGFR of CKD patients using EIT features and the age only is provided. The regression result is used to classify the CKD stage of the patient.

### 1. Materials and Methods

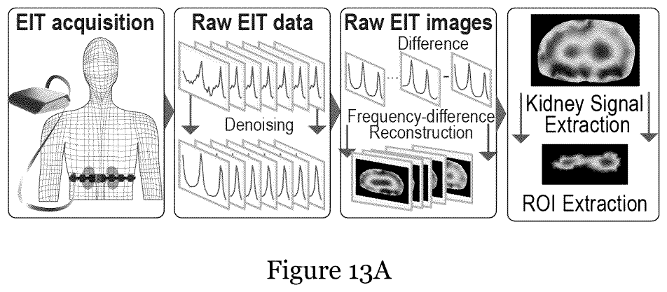

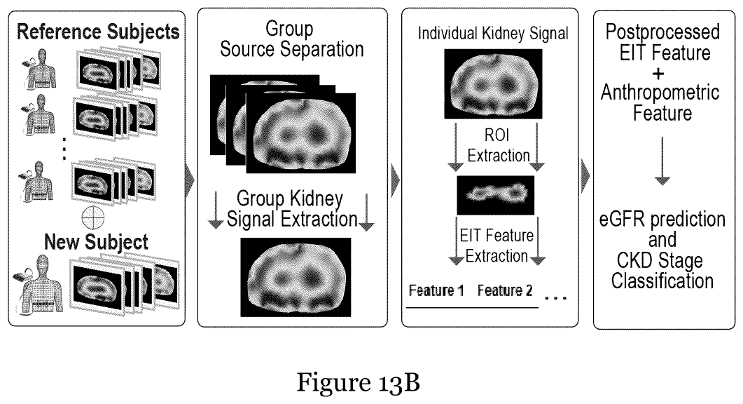

FIGS. 13A and 13B show an example operation including EIT data acquisition, processing, and analysis in this embodiment. Specifically, FIG. 13A shows an example of EIT data acquisition, data preprocessing and data analysis pipeline; FIG. 13B shows detailed illustration of eGFR prediction pipeline including the group source separation.

### 1.1 Subject

21 healthy individuals (controls) and 54 clinical diagnosed CKD patients are tested in this example. Corresponding demographics and physical characteristics, including gender, age, weight, height, and waist circumference for the subjects are collected. In this example, the subjects have undergone both eGFR measurement and EIT-kidney assessment.

### 1.2 EIT Acquisition

All EIT experiments in this example are conducted using a PVC electrode belt connected to a palm sized portable EIT console through an HDMI cable. The EIT console is connected to a computer with specialized software to collect, visualize and save the collected raw EIT data and system information for further data processing and analysis. The electrode belt consists of 16 modular electrode holders, each containing a printed circuit board that can be connected to 1 gel electrode. The electrode belt is placed circumferentially on the abdominal (upper abdominal) region. Throughout the whole EIT data acquisition process, the subjects are asked to breathe normally and to stay still.

In total, there are 208 (16 injection pairs×13 measurement pairs per injection pair) stimulation-measurement at each frequency. Each measurement is measured at a frame rate of 33 fps. 24 frequencies, in the range of 28 kHz to 300 kHz, are used in the EIT measurement and analysis process.

To acquire ground truth eGFRs, blood serum and urine samples are collected from each subject. Creatinine level, evaluated using the blood serum samples, is then used to derive the eGFR score of the subject with the known equation:

eGFR=141×min(S_{Cr}/κ,1)^{α}×max(S_{Cr}/κ,1)−1.209×0.993^{Age}×1.018_{[if female]}×1.159_{[if Black]}

where eGFR is the estimated glomerular filtration rate (mL/min/1.73 m²), S_{Cr} is the standardized serum creatinine (mg/dL), κ=0.7 for females or κ=0.9 for males, α=−0.329 for females or α=−0.411 for males, min indicates the minimum of S_{Cr}/κ or 1, max indicates the maximum of S_{Cr}/κ or 1, Age represents age of subject in years.

CKD stages are classified according to the value of eGFRs extracted from blood serum samples with the following criteria: Stage 1 CKD: (eGFR>90); Stage 2 CKD: (eGFR: 60-89); Stage 3 CKD: (eGFR: 30-60); Stage 4 CKD: (eGFR: 15-30); Stage 5 CKD: (eGFR: <15). The CKD stages are also grouped in terms of severity according to the following scheme: normal to mild (Stage 1-2), moderate (Stage 3) severe (Stage 4-5).

### 1.3 Data Pre-Processing

EIT reconstruction is conducted in Python using the library pyEIT and customized functions. Frequency difference EIT is used in this example, with reference frequency at 30 kHz and the other 23 frequencies ranging from 28 kHz to 300 kHz used for frequency difference reconstruction.

To ensure the quality of data for the purpose of further analysis, a measurement quality classifier and a reconstruction algorithm for arbitrary stimulation-measurement patterns are developed. For each frame, the classifier filters data to remove undesired effects due to uncontrollable events such as faulty measurements due to subject movements.

For each subject and each frequency, the mean of all frames is taken, all electrodes that are corrupted in any one of the frames are labelled and the corresponding stimulation-measurements are removed after taking the mean across frames. Since a reconstruction algorithm for arbitrary stimulation-measurement patterns is developed, we are able to reconstruct the conductivity images after the electrodes involved in faulty measurements are removed.

### 1.4 Data Analysis

Group source separation (FIG. 13B) is used to isolate signals from different internal tissues out of the reconstructed images of each individual. Frequency differences conductivity images at all contrast frequencies are used for the group source separation. Healthy subjects' data are used as reference group to assist in isolating the signals because the electrical responses from healthy subjects are more consistent in comparison to including all subjects from a variety of CKD stages, including subjects who experienced nephrotomy.

After isolating the signals sources, the source of the kidney signal is determined from the group separation result. From this group result, the individual kidney source is extracted. After the group source separation, the signal from the kidneys is the strongest amongst all other signals in the extracted kidney image component. The region of interest (ROI), i.e., the kidneys, is then extracted from the individual source.

After the kidney signal and the ROIs are extracted, EIT related features are generated, including but not limited to the mean conductivity within, outside the ROIs and the ratio between them. The data are split into train set and test set with 60 and 15 data points respectively in a stratified manner. A linear regression model is trained with the train set is evaluated using 5-fold cross validation.

Statistical comparisons are performed on CKD stages and severity classification using one-way ANOVA followed by multiple comparisons Bonferroni post-hoc tests (*p<0.05, **p<0.01, ***p<0.001).

The principle and operation of the group source separation has been described with reference to FIG. 2 hence is not repeated here.

### 2. Result

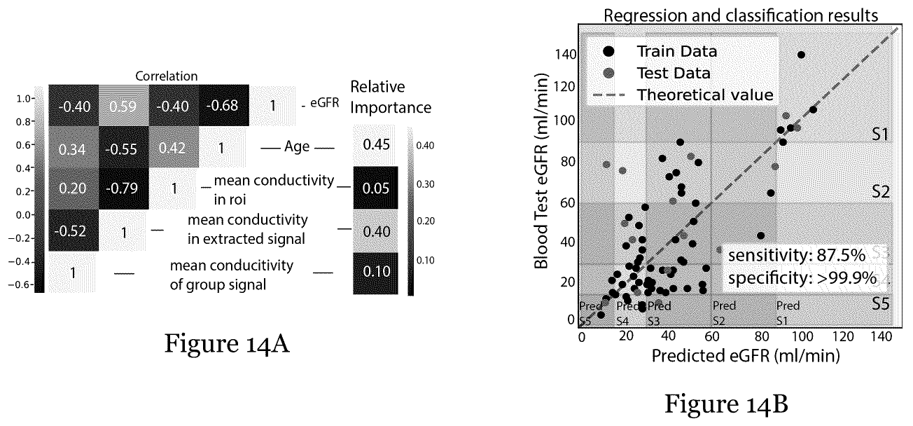

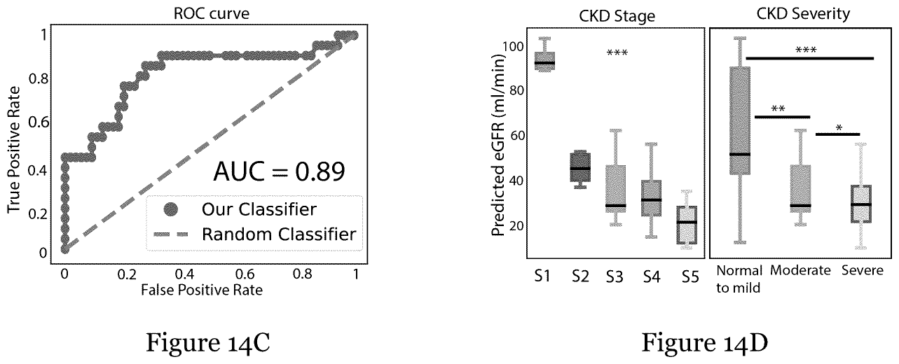

FIG. 14A shows the linear correlation coefficients and relative importance of EIT-features in the prediction of eGFR. FIG. 14B shows the correlation and classification specificity and sensitivity of the eGFR regression model. FIG. 14C shows a ROC curve of the regression model and the classification scheme. FIG. 14D shows comparison of EIT-predicted eGFR across CKD stages and CKD severity.

### 2.1 Correlation

It is found that the eGFR is correlated with mean conductivity of the group kidney source, the individual kidney source, and the kidney ROI with linear correlation coefficients of −0.4, 0.59 and −0.4. In addition to the EIT features, it is also found that the age is linear correlated to the eGFR with a coefficient of −0.68. It is found that the mean conductivity within the kidney ROIs is negatively linearly correlated to the mean conductivity of the individual kidney signal image with coefficient of −0.79. Please see FIG. 14A for details. Note that in FIG. 14A, the “mean conductivity in roi” refers to the mean of the ROI only in the conductivity map of the subject after group source separation (i.e., the lower image in the third box in FIG. 13B), the “mean conductivity in extracted signal” refers to the mean of the entire conductivity map of the subject after group source separation, and the “mean conductivity of group signal” refers to the mean of all conductivity maps of the subject and reference subjects after group source separation.

### 2.2 Regression Model

In this example the EIT features are fitted together with the age using a Lasso algorithm. The age has the highest relative importance of 0.45 among all the features, while the mean conductivity in the individual kidney source, in the group source, and in the ROI are 0.40, 0.10 and 0.05, respectively (FIG. 14A). The model has a cross-validated R² score of 0.4, which means that the EIT-feature prediction model has a significant correlation with the eGFR derived from blood serum and urine test.

### 2.3 CKD Classification

The CKD stages are obtained from the eGFR predicted from the regression model by the following criteria: Stage 1 CKD: (eGFR>90); Stage 2 CKD: (eGFR: 60-89); Stage 3 CKD: (eGFR: 30-60); Stage 4 CKD: (eGFR: 15-30); Stage 5 CKD: (eGFR: <15). Considering Stage 1 CKD as healthy and Stages 3, 4 and 5 CKD as non-healthy, a specificity of >99.9% and sensitivity of 87.5% in obtained (FIG. 14B). The area under curve (AUC) of the receiver operating characteristic (ROC) curve is found by setting the ground truth threshold eGFR for being healthy at 60 ml/min while varying the classification threshold for the classification from predicted eGFR. An AUC of 0.89 is obtained (FIG. 14C).

### 3. Discussion

This example demonstrates an eGFR estimation model and a CKD stage classification scheme using a portable, self-administrative EIT device. The operation of this device does not require dedicated professionally trained staff and clinical environment. Furthermore, this imaging device is non-invasive, ionizing-radiation-free and is cost-effective. This device can be used for medical screenings, for early chronic kidney disease diagnosis and longitudinal renal function monitoring without the need of public health services. Therefore, the device can enhance the quality and extend the area of application of telemedicine to renal function monitoring and chronic kidney diseases. Further, due to the portability and user-friendliness, the device can provide community-based CKD screening for individuals in locations that could be missed by public healthcare system.

In this example it is found that the mean conductivity in the individual extracted signal has a very negative linear correlation with the mean conductivity in ROI while having a relative importance of 0.4 in the Lasso model and a linear correlation coefficient of 0.59 with the eGFR. This suggests that the mean conductivity in the individual extracted signal is dominated by signals related to kidney functions.

The evaluation of classification specificity and sensitivity is based on 21 healthy subjects. A simulation based on the prediction error and a population eGFR distribution of the existing Lasso model is generated. Data from NHANES are used as the population eGFR distribution.

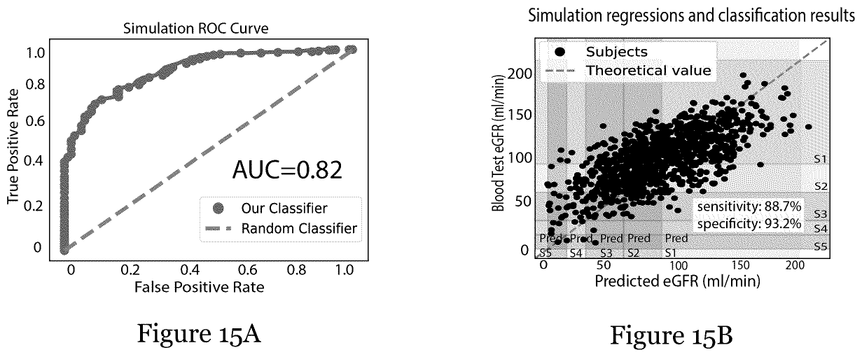

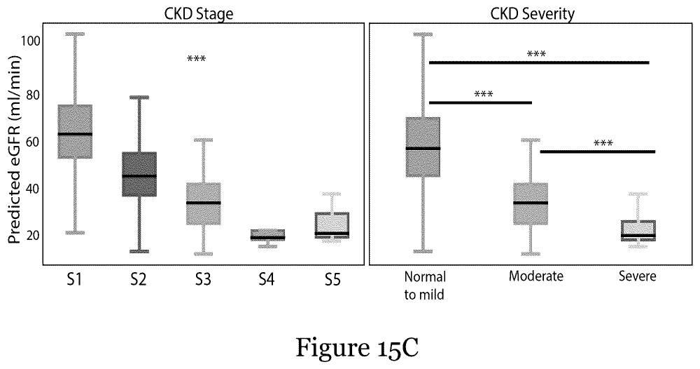

FIGS. 15A to 15C show the simulation results based on existing population eGFR distribution and the error of the proposed model in this example. Specifically, FIG. 15A shows an ROC curve of the simulation, FIG. 15B shows the correlation and classification specificity and sensitivity of simulation, FIG. 15C shows the comparison of the simulated EIT-based eGFR estimation across CKD stages and CKD severity.

An ROC curve with AUC=0.82 is obtained (FIG. 15A). The resulting sensitivity and specificity are 88.67% and 93.24% using N=1000 simulated data points (FIG. 15B).

In summary, in this example, clinical data and EIT data of 54 CKD patients and 21 healthy subjects are obtained with a portable EIT device. This example provides a data processing pipeline with a group source separation algorithm that isolates the kidney signals from raw EIT data. There is found significant correlations between standard eGFR and eGFR predicted from a linear model using EIT features and the age. CKD stages are classified from the estimated eGFR using the proposed model and 87.5% sensitivity and >99.9% specificity are obtained. This renal function assessment example demonstrates the feasibility of EIT to be used in the field of telemedicine as a non-invasive approach for early CKD diagnosis and potential for longitudinal CKD monitoring.

### EXAMPLE SYSTEMS

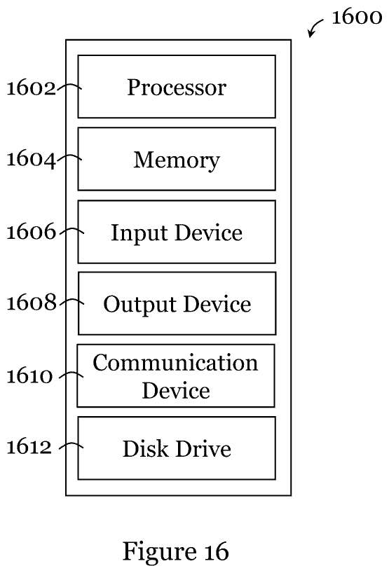

FIG. 16 shows an example data processing system 1600 that can be used to perform one or more of the method embodiments (partly or entirely) in some embodiments of the invention. The data processing system 1600 may be included in a server, a mobile device, a computer, etc. The data processing system 1600 generally includes suitable components necessary to receive, store, and execute appropriate computer instructions, commands, and/or codes. The main components of the data processing system 1600 are a processor 1602 and a memory (storage) 1604. The processor 1602 may include one or more: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries configured to interpret and/or to execute program instructions and/or to process signals and/or information and/or data. The processor 1602 can be used to perform machine learning based processing and non-machine learning based processing. The memory 1604 may include one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations. Appropriate computer instructions, commands, codes, information and/or data (e.g., any one or more of: EIT data, processed EIT data, fd-EIT data, specific organ/tissue-related components of fd-EIT data, one or more machine learning based models, anthropometric characteristic(s) of subjects, conductivity characteristic(s) associated with the subjects, quantitative or qualitative parameters associated with a health state or condition of respective organ(s)/tissue(s) of the subjects) may be stored in the memory 1604. Computer instructions for executing or facilitating executing the method embodiments of the invention may be stored in the memory 1604. The processor 1602 and memory (storage) 1604 may be integrated or separated (and operably connected). Optionally, the data processing system 1600 further includes one or more input devices 1606. Examples of such input device 1606 include: keyboard, mouse, stylus, image scanner, microphone, tactile/touch input device (e.g., touch sensitive screen), image/video input device (e.g., camera), etc. Optionally, the data processing system 1600 further includes one or more output devices 1608. Examples of such output device 1608 include: display (e.g., monitor, screen, projector, etc.), speaker, headphone, earphone, printer, additive manufacturing machine (e.g., 3D printer), etc. The display may include a LCD display, a LED/OLED display, or other suitable display, which may or may not be touch sensitive. The data processing system 1600 may further include one or more disk drives 1612 which may include one or more of: solid state drive, hard disk drive, optical drive, flash drive, magnetic tape drive, etc. A suitable operating system may be installed in the data processing system 1600, e.g., on the disk drive 1612 or in the memory 1604. The memory 1604 and the disk drive 1612 may be operated by the processor 1602. Optionally, the data processing system 1600 also includes a communication device 1610 for establishing one or more communication links (not shown) with one or more other computing devices, such as servers, personal computers, terminals, tablets, phones, watches, IoT devices, or other wireless computing devices. The communication device 1610 may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection, or other wired or wireless communication interfaces. Transceiver may be implemented by one or more devices (integrated transmitter(s) and receiver(s), separate transmitter(s) and receiver(s), etc.). The communication link(s) may be wired or wireless for communicating commands, instructions, information and/or data. In one example, the processor 1602, the memory 1604 (optionally the input device(s) 1606, the output device(s) 1608, the communication device(s) 1610 and the disk drive(s) 1612, if present) are connected with each other, directly or indirectly, through a bus, a Peripheral Component Interconnect (PCI), such as PCI Express, a Universal Serial Bus (USB), an optical bus, or other like bus structure. In one embodiment, at least some of these components may be connected wirelessly, e.g., through a network, such as the Internet or a cloud computing network. A person skilled in the art would appreciate that the data processing system 1600 shown in FIG. 16 is merely an example and that the data processing system 1600 can in other embodiments have different configurations (e.g., include additional components, has fewer components, etc.).

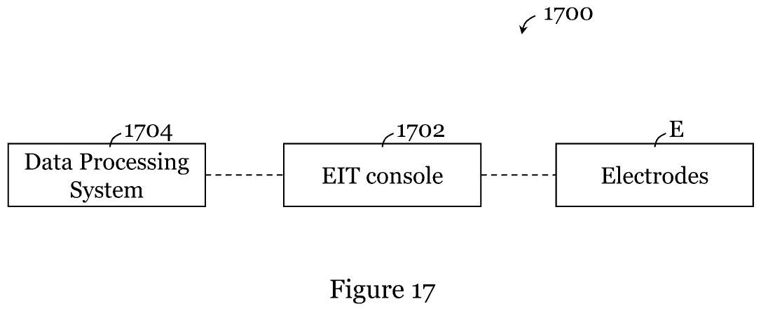

FIG. 17 shows an EIT system 1700 that can be used for EIT data acquisition and processing in some embodiments of the invention. The EIT system 1700 generally includes an EIT console 1702 (which may or may not be portable), electrodes E electrically connected with the EIT console 1702 to obtain signals from the subject, and a data processing system 1704 operably connected with the EIT console 1702. In one example, the EIT console 1702 is mainly used to control signal transmission and receive, to and from the body of the subject, via the electrodes E. In one example, the data processing system 1704 is mainly used to process the signals obtained by the EIT console 1702 via the electrodes E. In one example, the data processing system 1704 may be at least partly integrated with the EIT console such that the data processing can be at least partly performed at the EIT console. In one example, the data processing system 1704 may be the system 1600 of FIG. 16.

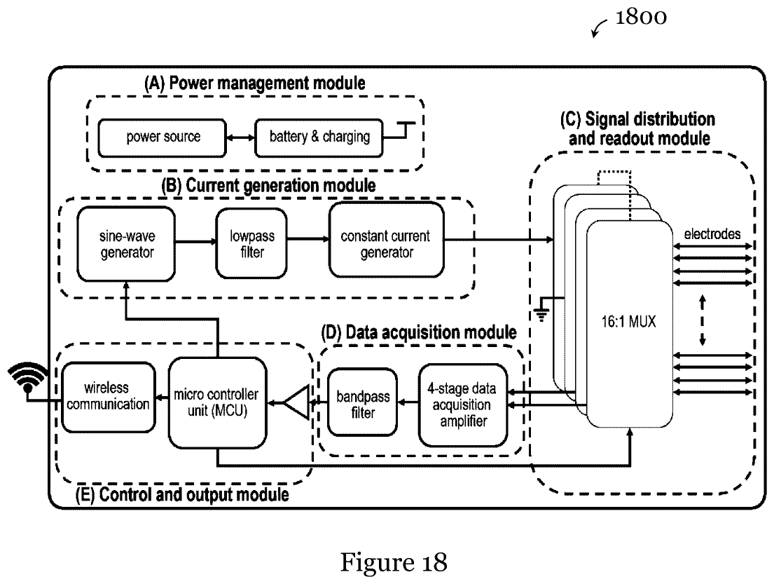

FIG. 18 shows example EIT console 1800 that can be used for EIT data acquisition in some embodiments of the invention. In this example the EIT console is portable. Referring to FIG. 18, the EIT console 1800 generally includes a power management module for constant power supply, a current generation module for alternating current generation, a signal distribution and readout module for current injection and voltage readout, a data acquisition module for potential difference measurement, amplification and acquisition, and a control and output module for module coordination, data processing and cloud-server communication. As illustrated in FIG. 18, the EIT console 1800 in this example includes an EIT console having 5 major modules:
Power management module that provides power supply to all other modules through the power socket or battery.
Current generation module that primarily includes a sine wave generator and a constant current generator successively to generate an alternating current of 1 mApp and a voltage amplitude of 1 Vpp. A low-pass filter may be included to suppress total harmonic distortion and ambient electromagnetic interference (e.g., power line noise).
Signal distribution and readout module that introduces the generated current to the subject via the 16-electrode belt using a set of CMOS multiplexers (MUXs). Four MUXs are used, in which two MUXs are employed for current injection and the other two for voltage readout. The MUXs are configured into the adjacent-scan pattern through the microcontroller unit (MCU).
Data acquisition module that is the analog front-end (AFE) that acquires, measures and amplifies the potential differences from the electrodes. The AFE comprises a four-stage wide input differential amplifier with high common-mode rejection ratio (CMRR), and a bandpass filter.
Control and output module that includes an analog-to-digital converter (ADC), a MCU and a wireless communication chip. The potential differences obtained from the data acquisition module are digitized by the ADC, processed in the MCU unit, and transferred to an external device (server, phone, computer, etc.) for image reconstruction and processing.

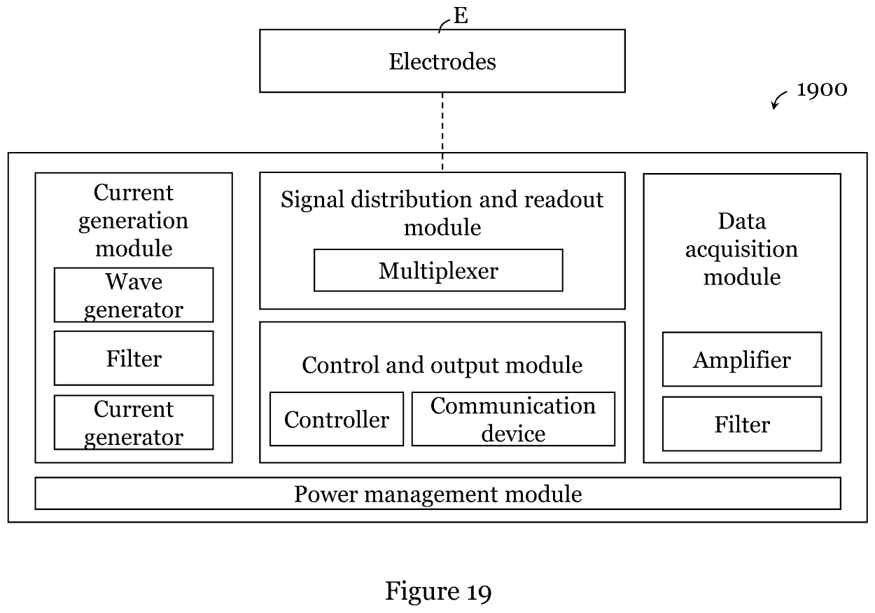

FIG. 19 is a block diagram of another example EIT console 1900 in some embodiments of the invention. The EIT console 1900 is basically a generalized version of the EIT console 1800. The EIT console 1900 may or may not be portable.

Although not required, the embodiments described with reference to the Figures can be implemented as an application programming interface (API) or as a series of libraries for use by a developer or can be included within another software application, such as a terminal or computer operating system or a portable computing device operating system. Generally, as program modules include routines, programs, objects, components and data files assisting in the performance of particular functions, the skilled person will understand that the functionality of the software application may be distributed across a number of routines, objects and/or components to achieve the same functionality desired herein.

It will also be appreciated that where the methods and systems of the invention are either wholly implemented by computing system or partly implemented by computing systems then any appropriate computing system architecture may be utilized. This will include stand-alone computers, network computers, dedicated or non-dedicated hardware devices. Where the terms “computing system” and “computing device” are used, these terms are intended to include (but not limited to) any appropriate arrangement of computer or information processing hardware capable of implementing the function described.

It will be appreciated by a person skilled in the art that variations and/or modifications may be made to the described and/or illustrated embodiments of the invention to provide other embodiments of the invention. The described/or illustrated embodiments of the invention should therefore be considered in all respects as illustrative, not restrictive. Example optional features of the invention are provided in the summary and the description. Some embodiments of the invention may include one or more of these optional features (some of which are not specifically illustrated in the drawings). Some embodiments of the invention may lack one or more of these optional features (some of which are not specifically illustrated in the drawings). For example, the method embodiments of the invention are not limited for use in humans but can be use in other animals. The method can be applied for determining health state or condition of different tissues or organs of different subjects. The EIT data acquisition need not be performed using a portable EIT device such as the ones illustrated—the EIT data can be obtained using other EIT devices. The data processing methods of the invention can be implemented on any suitable device or devices (including one or more of server, computer, phone, the EIT console (portable or not), etc.). For example, the method of the invention can be used for diagnosis for different diseases associated with different tissues or organs of interest.

1. A computer-implemented method, comprising:
processing a EIT data set of a subject to determine one or more conductivity characteristics associated with a tissue or organ of the subject; and
determining, based on at least the one or more determined conductivity characteristics, a health state or condition of the tissue or organ of the subject.

2. The computer-implemented method of claim 1, wherein the determining comprises:
determining, based on at least the one or more determined conductivity characteristics, whether the subject has a disease associated with the tissue or organ, and optionally: further classifying a stage or a severity of the disease associated with the tissue or organ.

3. The computer-implemented method of claim 1, wherein the determining comprises:
processing, at least, the one or more determined conductivity characteristics of the subject and one or more anthropometric characteristics of the subject, using a machine learning based processing model, to determine a quantitative or qualitative parameter associated with the health state or condition of the tissue or organ of the subject.

4. The computer-implemented method of claim 1, wherein the determining comprises:
processing, using a machine learning based processing model,
(i) the one or more determined conductivity characteristics of the subject,
(ii) one or more anthropometric characteristics of the subject, and
(iii) one or more determined conductivity characteristics of one or more reference subjects and/or of a group containing the subject and the one or more reference subjects,
to determine a quantitative or qualitative parameter associated with the health state or condition of the tissue or organ of the subject.

5. The computer-implemented method of claim 3, wherein the machine learning based processing model comprises a regression model.

6. The computer-implemented method of claim 3, wherein the machine learning based processing model comprises a classification model.

7. The computer-implemented method of claim 6, wherein the one or more anthropometric characteristics comprise, or are related to, one or more of: age of the subject, weight of the subject, height of the subject, waist circumference of the subject, waist-over-height ratio of the subject, body mass index (BMI) of the subject, gender of the subject, and race of the subject.

8. The computer-implemented method of claim 7, wherein the quantitative or qualitative parameter associated with the health state or condition of the tissue or organ of the subject comprises: a value associated with an estimated performance of the tissue or organ of the subject.

9. The computer-implemented method of claim 8, wherein the determining further comprises:
comparing the quantitative or qualitative parameter associated with the health state or condition of the tissue or organ of the subject with reference parameter data to determine whether the subject has a disease associated with the tissue or organ.

10. The computer-implemented method of claim 9, wherein the determining further comprises:
classifying, based on the comparing, a stage or a severity of the disease associated with the tissue or organ.

11. The computer-implemented method of claim 10,
wherein the EIT data set contains EIT data obtain from a region of the subject containing the tissue or organ;
wherein the EIT data set is obtained by
(a) providing excitation signals at a frequency to the subject via electrodes attached to the region of the subject,
(b) measuring responsive signals received via the electrodes as a result of the providing of the excitation signals, and
(c) repeating steps (a) and (b) for a plurality of frequencies; and
wherein the EIT data set comprises a plurality of EIT data subsets each associated with a respective one of the plurality of frequencies.

12. The computer-implemented method of claim 11, wherein the processing comprises:
(i) processing the EIT data set to obtain a frequency difference EIT data set, the frequency difference EIT data set includes a plurality of frequency difference EIT data subsets;
(ii) performing a group source separation operation using the frequency difference EIT data set and one or more reference frequency difference EIT data sets of corresponding one or more reference subjects to determine component of the frequency difference EIT data set related to the tissue or organ and component of each of the one or more reference frequency difference EIT data sets related to the tissue or organ; and
(iii) performing a conductivity characteristics extraction operation using the component of the frequency difference EIT data set related to the tissue or organ and optionally the component of each of the one or more reference frequency difference EIT data sets related to the tissue or organ to determine at least the one or more conductivity characteristics of the subject.

13. The computer-implemented method of claim 12, wherein the processing further comprises:
pre-processing the EIT data set before the processing in (i) so that the EIT data set processed in (i) is a pre-processed EIT data set.

14. The computer-implemented method of claim 13, wherein the pre-processing of the EIT data set comprises:
filtering and/or smoothing each of the plurality of EIT data subsets.

15. The computer-implemented method of claim 14, wherein the pre-processing of the EIT data set comprises:
processing the EIT data set using a classifier model to determine respective performance of each of the plurality of electrodes, the performance being associated with quality of responsive signals or data obtained from the respective electrode; and
preventing the responsive signals or data obtained via any one or more of the plurality of electrodes determined to have insufficient performance from being included in the processed EIT data set.

16. The computer-implemented method of claim 15, wherein the processing of the EIT data set in (ii) comprises:
determining, for each respective one or more of the plurality of processed EIT data subsets, respective difference between the respective processed EIT data subset and a reference EIT data subset, so as to obtain the plurality of frequency difference EIT data subsets each associated with a respective one of a difference between the respective processed EIT data subset and a reference EIT data subset.

17. The computer-implemented method of claim 16, wherein the reference EIT data subset comprises at least one of the plurality of processed EIT data subsets.

18. The computer-implemented method of claim 17, wherein the performing of the group source separation operation comprises:
performing a dimensionality reduction operation on the frequency difference EIT data set and one or more reference frequency difference EIT data sets of corresponding one or more reference subjects.

19. The computer-implemented method of claim 18, wherein the performing of the conductivity characteristics extraction operation comprises:
determining, using the component of the frequency difference EIT data set related to the tissue or organ, the one or more conductivity characteristics of the subject.

20. The computer-implemented method of claim 19, wherein the performing of the conductivity characteristics extraction operation comprises:
determining, using the component of the frequency difference EIT data set related to the tissue or organ and respective component of each of the one or more reference frequency difference EIT data sets related to the tissue or organ, one or more conductivity characteristics of a group containing the subject and the one or more reference subjects; and
wherein the determining of the health state or condition of the tissue or organ of the subject is further based on the one or more conductivity characteristics of the group.

21. The computer-implemented method of claim 1, wherein the tissue or organ comprises a lung, a kidney, a liver, or a heart.

22. (canceled)

23. (canceled)

24. (canceled)
